(19) United States
(12) Patent Application Publication
(10) Pub. No.: US 2015/0105269 A1
(43) Pub. Date: Apr. 16, 2015

(54) BIOMARKERS FOR INCREASED RISK OF DRUG-INDUCED OSTEONECROSIS OF THE JAW

(71) Applicant: Severe Adverse Event (SAE) Consortium, Chicago, IL (US)

(72) Inventor: Aris FLORATOS, Astoria, NY (US)

(21) Appl. No.: 14/512,192

(22) Filed: Oct. 10, 2014

Related U.S. Application Data
(60) Provisional application No. 61/889,468, filed on Oct. 10, 2013.

(51) Int. Cl. C12Q 1/68

(52) U.S. Cl. CPC C12Q 1/6883; C12Q 2600/156; C12Q 2600/106
USPC 506/2; 435/6.11; 506/9

(57) ABSTRACT

The present disclosure provides a method for predicting the risk of a patient for developing adverse drug reactions, particularly drug-induced osteonecrosis of the jaw (ONJ). The disclosure also provides a method of identifying a subject afflicted with, or at risk of, developing ONJ. In some aspects, the methods comprise analyzing at least one genetic marker, wherein the presence of the at least one genetic marker indicates that the subject is afflicted with, or at risk of, developing ONJ.

This application claims the benefit of priority of U.S. Provisional Application Ser. No. 61/889,468, filed Oct. 10, 2013, the contents of which are hereby incorporated by reference in its entirety.

### TECHNICAL FIELD

The present disclosure relates generally to methods for identifying genetic risk factors for adverse reactions to drugs. More specifically, the present disclosure relates to methods for predicting what drugs will cause osteonecrosis of the jaw, and in which patients.

### BACKGROUND

Adverse reactions to drugs are a major cause of morbidity and death. Frequently occurring adverse drug reactions include osteonecrosis of the jaw (ONJ). ONJ is a severe bone disease that can affect the upper jaw (maxilla) and/or lower jaw (mandible); however, the mandible is reportedly more susceptible to ONJ. ONJ can be described as death of bone or bone marrow resulting from persistent ischemia and inadequate access to nutrients. Clinical symptoms of ONJ include localized pain and neuropathy; erythema, swelling, and/or inflammation of soft tissue (e.g., the gums); suppuration; halitosis; loosening of previously stable teeth; bone inflammation, infection, and/or fracture; and exposure of maxillary and/or mandibular bone through lesions that do not heal (i.e., last for more than about eight weeks). ONJ-related lesions can develop following dental procedures (e.g., extraction) but can also occur spontaneously. Based on severity, number of lesions, and lesion size, ONJ is classified into four grades ranging from asymptomatic (e.g., one lesion<about 0.5 cm) to severe (e.g., multiple lesions>2.0 cm). In severe cases, the affected bone may be surgically removed.

ONJ is associated with cancer treatments (including radiation), infection, steroid use, and potent antiresorptive therapies that help prevent the loss of bone mass. Examples of potent antiresorptive therapies include bisphosphonates, which are used to treat osteoporosis, osteitis deformans (Paget's disease of the bone), bone cancers, and other conditions that lead to bone fragility. Bisphosphonate-related osteonecrosis of the jaw (BRONJ) has been associated with the bisphosphonates alendronate, pamidronate, zoledronate, risedronate, ibandronate, and denosumab. The risk of BRONJ may depend on the dose of medication, the length of therapy, and the medical condition for which the bisphosphonate is prescribed. As a result, cancer patients taking higher doses of bisphosphonates, particularly intravenously, may be at a higher risk. ONJ has also been associated with use of steroids, particularly corticosteroids, including glucocorticoids. For example, patients taking dexamethasone and other glucocorticoids may be at increased risk of ONJ.

There is a need for markers that can predict the existence of or predisposition to ONJ. Several studies have identified genetic risk factors for drug-related severe adverse events. However, there is currently no clinically useful method for predicting what drugs will cause ONJ, and in which patients.

### SUMMARY

An aspect of the invention provides a method for predicting the risk of a patient for developing adverse drug reactions, particularly osteonecrosis of the jaw (ONJ).

ONJ may be caused by drugs such as steroids and potent antiresorptive therapies including bisphosphonates.

Another aspect of the invention provides a method of identifying a subject afflicted with, or at risk of, developing ONJ comprising (a) obtaining a nucleic acid-containing sample from the subject; and (b) analyzing the sample to detect the presence of at least one genetic marker, wherein the presence of the at least one genetic marker indicates that the subject is afflicted with, or at risk of, developing ONJ. The method may further comprise treating the subject based on the results of step (b). The method may further comprise taking a clinical history from the subject. Genetic markers that are useful for the invention include, but are not limited to, alleles, microsatellites, SNPs, and haplotypes. The sample may be any sample capable of being obtained from a subject, including but not limited to blood, sputum, saliva, mucosal scraping and tissue biopsy samples.

In some embodiments of the invention, the genetic markers are SNPs selected from those listed in Tables 1-5. In other embodiments, genetic markers that are linked to each of the SNPs can be used to predict the corresponding ONJ risk.

The presence of the genetic marker can be detected using any method known in the art. Analysis may comprise nucleic acid amplification, such as PCR. Analysis may also comprise primer extension, restriction digestion, sequencing, hybridization, a DNAse protection assay, mass spectrometry, labeling, and separation analysis.

Other features and advantages of the disclosure will be apparent from the detailed description, drawings and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a Manhattan plot that summarizes the WGA study result for the ONJ vs. controls cohort. Each dot in the plot represents an SNP, the x-axis refers to its position on chromosomes (human NCBI build 36), and the y-axis refers to the −log 10 (p-value) from the case/control study.

FIG. 2 is a Manhattan plot that summarizes the WGA study result for the spontaneous ONJ vs. controls cohort. Each dot in the plot represents an SNP, the x-axis refers to its position on chromosomes (human NCBI build 36), and the y-axis refers to the −log 10 (p-value) from the case/control study.

FIG. 3 is a Manhattan plot that summarizes the WGA study result for the ONJ vs. controls cohort. Each dot in the plot represents an SNP, the x-axis refers to its position on chromosomes (human NCBI build 36), and the y-axis refers to the −log 10 (p-value) from the case/control study.

FIG. 4 is a Manhattan plot that summarizes the WGA study result for alendronate specific ONJ vs. controls cohort. Each dot in the plot represents an SNP, the x-axis refers to its position on chromosomes (human NCBI build 36), and the y-axis refers to the −log 10 (p-value) from the case/control study.

FIG. 5 is a Manhattan plot that summarizes the WGA study result for Dimensions ONJ vs. controls cohort. Each dot in the plot represents an SNP, the x-axis refers to its position on chromosomes (human NCBI build 36), and the y-axis refers to the −log 10 (p-value) from the case/control study.

FIG. 6 is a Manhattan plot that summarizes the WGA study result for latency ONJ vs. controls cohort. Each dot in the plot represents an SNP, the x-axis refers to its position on chromosomes (human NCBI build 36), and the y-axis refers to the −log 10 (p-value) from the case/control study.

FIG. 7 is a Manhattan plot that summarizes the WGA study result for spontaneous ONJ vs. controls cohort. Each dot in the plot represents an SNP, the x-axis refers to its position on chromosomes (human NCBI build 36), and the y-axis refers to the −log 10 (p-value) from the case/control study.

FIG. 8 is a Manhattan plot that summarizes the WGA study result for zoledronate specific ONJ vs. controls cohort. Each dot in the plot represents an SNP, the x-axis refers to its position on chromosomes (human NCBI build 36), and the y-axis refers to the −log 10 (p-value) from the case/control study.

### DETAILED DESCRIPTION

For the purposes of promoting an understanding of the principles of the invention, reference will now be made to specific embodiments and specific language will be used to describe the same. It will nevertheless be understood that no limitation of the scope of the invention is thereby intended, and that such alterations and further modifications of the invention, and such further applications of the principles of the invention as illustrated herein as would normally occur to one skilled in the art to which the invention relates, are contemplated as within the scope of the invention.

All terms as used herein are defined according to the ordinary meanings they have acquired in the art. Such definitions can be found in any technical dictionary or reference known to the skilled artisan, such as the McGraw-Hill Dictionary of Scientific and Technical Terms (McGraw-Hill, Inc.), Molecular Cloning: A Laboratory Manual (Cold Springs Harbor, N.Y.), Remington's Pharmaceutical Sciences (Mack Publishing, PA), and Stedman's Medical Dictionary (Williams and Wilkins, MD). These references, along with those references, patents, and patent applications cited herein are hereby incorporated by reference in their entirety.

The term “marker” as used herein refers to any morphological, biochemical, or nucleic acid-based phenotypic difference which reveals a DNA polymorphism. The presence of markers in a sample may be useful to determine the phenotypic status of a subject (e.g., whether an individual has or has not been afflicted with ONJ), or may be predictive of a physiological outcome (e.g., whether an individual is likely to develop ONJ). The markers may be differentially present in a biological sample or fluid, such as blood plasma or serum. The markers may be isolated by any method known in the art, including methods based on mass, binding characteristics, or other physicochemical characteristics. As used herein, the term “detecting” includes determining the presence, the absence, or a combination thereof, of one or more markers.

Non-limiting examples of nucleic acid-based, genetic markers include alleles, microsatellites, single nucleotide polymorphisms (SNPs), haplotypes, copy number variants (CNVs), insertions, and deletions.

The term “allele” as used herein refers to an observed class of DNA polymorphism at a genetic marker locus. Alleles may be classified based on different types of polymorphism, for example, DNA fragment size or DNA sequence. Individuals with the same observed fragment size or same sequence at a marker locus have the same genetic marker allele and thus are of the same allelic class.

The term “locus” as used herein refers to a genetically defined location for a collection of one or more DNA polymorphisms revealed by a morphological, biochemical or nucleic acid-bred analysis.

The term “genotype” as used herein refers to the allelic composition of an individual at genetic marker loci under study, and “genotyping” refers to the process of determining the genetic composition of individuals using genetic markers.

The term “single nucleotide polymorphism” (SNP) as used herein refers to a DNA sequence variation occurring when a single nucleotide in the genome or other shared sequence differs between members of a species or between paired chromosomes in an individual. The difference in the single nucleotide is referred to as an allele. A “haplotype” as used herein refers to a set of single SNPs on a single chromatid that are statistically associated.

The term “microsatellite” as used herein refers to polymorphic loci present in DNA that comprise repeating units of 1-6 base pairs in length.

An aspect of the invention provides a method for predicting the risk of a patient for developing adverse drug reactions, particularly osteonecrosis of the jaw (ONJ). As used herein, an “adverse drug reaction” is as an undesired and unintended effect of a drug. A “drug” as used herein is any compound or agent that is administered to a patient for prophylactic, diagnostic or therapeutic purposes.

ONJ may be caused by many different classes of drugs. Nonlimiting examples of drugs known to cause ONJ include potent antiresorptive therapies, such as the bisphosphonates alendronate, pamidronate, zoledronate, risedronate, ibandronate, and denosumab, as well as steroids, such as dexamethasone and other glucocorticoids.

Another aspect of the invention provides a method of identifying a subject afflicted with or at risk of developing ONJ comprising (a) obtaining a nucleic acid-containing sample from the subject; and (b) analyzing the sample to detect the presence of at least one genetic marker, wherein the presence of the at least one genetic marker indicates that the subject is afflicted with or at risk of developing ONJ. The method may further comprise treating the subject based on the results of step (b). The method may further comprise taking a clinical history from the subject. Genetic markers that are useful for the invention include, but are not limited to, alleles, microsatellites, SNPs, haplotypes, CNVs, insertions, and deletions.

In some embodiments of the invention, the genetic markers are one or more SNPs selected from those listed in Tables 1-5. The reference numbers provided for these SNPs are from the NCBI SNP database, at www.ncbi.nlm.nih.gov/entrez/query.fcgi?db=snp.

Each person's genetic material contains a unique SNP pattern that is made up of many different genetic variations. SNPs may serve as biological markers for pinpointing a disease on the human genome map, because they are usually located near a gene found to be associated with a certain disease. Occasionally, a SNP may actually cause a disease and, therefore, can be used to search for and isolate the disease-causing gene.

In accordance with the present disclosure, at least one marker may be detected. It is to be understood, and is described herein, that one or more markers may be detected and subsequently analyzed, including several or all of the markers identified. Further, it is to be understood that the failure to detect one or more of the markers of the invention, or the detection thereof at levels or quantities that may correlate with ONJ, may be useful as a means of selecting the individuals afflicted with or at risk for developing ONJ, and that the same forms a contemplated aspect of the invention.

In addition to the SNPs listed in Tables 1-5, genetic markers that are linked to each of the SNPs may be used to predict the corresponding ONJ risk as well. The presence of equivalent genetic markers may be indicative of the presence of the allele or SNP of interest, which, in turn, is indicative of a risk for ONJ. For example, equivalent markers may co-segregate or show linkage disequilibrium with the marker of interest. Equivalent markers may also be alleles or haplotypes based on combinations of SNPs.

The equivalent genetic marker may be any marker, including alleles, microsatellites, SNPs, and haplotypes. In some embodiments, the useful genetic markers are about 200 kb or less from the locus of interest. In other embodiments, the markers are about 100 kb, 80 kb, 60 kb, 40 kb, or 20 kb or less from the locus of interest.

To further increase the accuracy of risk prediction, the marker of interest and/or its equivalent marker may be determined along with the markers of accessory molecules and co-stimulatory molecules which are involved in the interaction between antigen-presenting cell and T-cell interaction. For example, the accessory and co-stimulatory molecules include cell surface molecules (e.g., CD80, CD86, CD28, CD4, CD8, T cell receptor (TCR), ICAM-1, CD11a, CD58, CD2, etc.), and inflammatory or pro-inflammatory cytokines, chemokines (e.g., TNF-α), and mediators (e.g., complements, apoptosis proteins, enzymes, extracellular matrix components, etc.). Also of interest are genetic markers of drug metabolizing enzymes which are involved in the bioactivation and detoxification of drugs. Non-limiting examples of drug metabolizing enzymes include phase I enzymes (e.g., cytochrome P450 superfamily), and phase II enzymes (e.g., microsomal epoxide hydrolase, arylamine N-acetyltransferase, UDP-glucuronosyl-transferase, etc.).

Another aspect of the invention provides a method for pharmacogenomic profiling. Accordingly, a panel of genetic factors is determined for a given individual, and each genetic factor is associated with the predisposition for a disease or medical condition, including adverse drug reactions. In some embodiments, the panel of genetic factors may include at least one SNP selected from Tables 1-5. The panel may include equivalent markers to the markers in Tables 1-5. The genetic markers for accessory molecules, co-stimulatory molecules and/or drug metabolizing enzymes described above may also be included.

Yet another aspect of the invention provides a method of screening and/or identifying agents that can be used to treat ONJ by using any of the genetic markers of the invention as a target in drug development. For example, cells expressing any of the SNPs or equivalents thereof may be contacted with putative drug agents, and the agents that bind to the SNP or equivalent are likely to inhibit the expression and/or function of the SNP. The efficacy of the candidate drug agent in treating ONJ may then be further tested.

In some embodiments, it may be useful to amplify the target sequence before evaluating the genetic marker. Nucleic acids used as a template for amplification may be isolated from cells, tissues or other samples according to standard methodologies such as are described, for example, in Sambrook et al., 1989. In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA. The DNA also may be from a cloned source or synthesized in vitro.

The term “primer,” refers to any nucleic acid that is capable of priming the synthesis of a nascent nucleic acid in a template-dependent process. Typically, primers are oligonucleotides from ten to twenty or thirty base pairs in length, but longer sequences can be employed. Primers may be provided in double-stranded or single-stranded form.

For amplification of SNPs, pairs of primers designed to selectively hybridize to nucleic acids flanking the polymorphic site may be contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex may be contacted with one or more enzymes that facilitate template-dependent nucleic acid synthesis. Multiple rounds of amplification, also referred to as “cycles,” are conducted until a sufficient amount of amplification product is produced.

It is also possible that multiple target sequences will be amplified in a single reaction. Primers designed to expand specific sequences located in different regions of the target genome, thereby identifying different polymorphisms, would be mixed together in a single reaction mixture. The resulting amplification mixture would contain multiple amplified regions, and could be used as the source template for polymorphism detection using the methods described in this application.

Any known template dependent process may be advantageously employed to amplify the oligonucleotide sequences present in a given template sample. One of the best known amplification methods is the polymerase chain reaction (PCR), which is described in U.S. Pat. Nos. 4,683,195, 4,683,202 and 4,800,159, and in Innis et al., 1988, each of which is incorporated herein by reference in their entirety.

A reverse transcriptase PCR amplification procedure may be performed when the source of nucleic acid is fractionated or whole cell RNA. Methods of reverse transcribing RNA into cDNA are well known and are described in, for example, Sambrook et al., 1989. Alternative exemplary methods for reverse polymerization utilize thermostable DNA polymerases. These methods are described, for example, in International Publication WO 90/07641. Polymerase chain reaction methodologies are well known in the art. Representative methods of RT-PCR are described, for example, in U.S. Pat. No. 5,882,864.

Another method for amplification is ligase chain reaction (LCR), disclosed, for example, in European Application No. 320 308, incorporated herein by reference in its entirety. U.S. Pat. No. 4,883,750 describes a method similar to LCR for binding probe pairs to a target sequence. A method based on PCR and oligonucleotide ligase assay (OLA), disclosed, for example, in U.S. Pat. No. 5,912,148, may also be used.

Another ligase-mediated reaction is disclosed by Guilfoyle et al. (1997). Genomic DNA is digested with a restriction enzyme and universal linkers are then ligated onto the restriction fragments. Primers to the universal linker sequence are then used in PCR to amplify the restriction fragments. By varying the conditions of the PCR, one can specifically amplify fragments of a certain size (e.g., fewer than 1000 bases). A benefit to using this approach is that each individual region would not have to be amplified separately. There would be the potential to screen thousands of SNPs from the single PCR reaction.

Q-beta Replicase, described, for example, in International Application No. PCT/US87/00880, may also be used as an amplification method in the present disclosure. In this method, a replicative sequence of RNA that has a region complementary to that of a target is added to a sample in the presence of an RNA polymerase. The polymerase will copy the replicative sequence, which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present disclosure (Walker et al., 1992). Strand Displacement Amplification (SDA), disclosed, for example, in U.S. Pat. No. 5,916,779, is another method of carrying out isothermal amplification of nucleic acids which involves multiple rounds of strand displacement and synthesis, e.g., nick translation.

Other nucleic acid amplification procedures include polymerization-based amplification systems (TAS), for example, nucleic acid sequence based amplification (NASBA) and 3SR (Kwoh et al., 1989; International Application WO 88/10315, incorporated herein by reference in their entirety). European Application No. 329 822 discloses a nucleic acid amplification process involving cyclically synthesizing single-stranded RNA (ssRNA), ssDNA, and double-stranded DNA (dsDNA), which may be used in accordance with the present disclosure.

International Application WO 89/06700 discloses a nucleic acid sequence amplification scheme based on the hybridization of a promoter region/primer sequence to a target single-stranded DNA (ssDNA) followed by polymerization of many RNA copies of the sequence. This scheme is not cyclic, i.e., new templates are not produced from the resultant RNA transcripts. Other amplification methods include “race” and “one-sided PCR” (Frohman, 1990; Ohara et al., 1989).

### Methods of Detection

The genetic markers of the invention may be detected using any method known in the art. For example, genomic DNA may be hybridized to a probe that is specific for the allele of interest. The probe may be labeled for direct detection, or contacted by a second, detectable molecule that specifically binds to the probe. Alternatively, cDNA, RNA, or the protein product of the allele may be detected. For example, serotyping or microcytotoxity methods may be used to determine the protein product of the allele. Similarly, equivalent genetic markers may be detected by any methods known in the art.

It is within the purview of one of skill in the art to design genetic tests to screen for ONJ or a predisposition for ONJ based on analysis of the genetic markers of the invention. For example, a genetic test may be based on the analysis of DNA for SNP patterns. Samples may be collected from a group of individuals affected by ONJ due to drug treatment and the DNA analyzed for SNP patterns. Non-limiting examples of sample sources include blood, sputum, saliva, mucosal scraping or tissue biopsy samples. These SNP patterns may then be compared to patterns obtained by analyzing the DNA from a group of individuals unaffected by ONJ due to drug treatment. This type of comparison, called an “association study,” can detect differences between the SNP patterns of the two groups, thereby indicating which pattern is most likely associated with ONJ. Eventually, SNP profiles that are characteristic of a variety of diseases will be established. These profiles can then be applied to the population at general, or those deemed to be at particular risk of developing ONJ.

Various techniques may be used to assess genetic markers. Non-limiting examples of a few of these techniques are discussed here and also described in US Patent Publication 2007/026827, the disclosure of which is herein incorporated by reference in its entirety. In accordance with the present disclosure, any of these methods may be used to design genetic tests for affliction with or predisposition to ONJ. Additionally, these methods are continually being improved and new methods are being developed. It is contemplated that one of skill in the art will be able to use any improved or new methods, in addition to any existing method, for detecting and analyzing the genetic markers of the invention.

Restriction Fragment Length Polymorphism (RFLP) is a technique in which different DNA sequences may be differentiated by analysis of patterns derived from cleavage of that DNA. If two sequences differ in the distance between sites of cleavage of a particular restriction endonuclease, the length of the fragments produced will differ when the DNA is digested with a restriction enzyme. The similarity of the patterns generated can be used to differentiate species (and even individual species members) from one another.

Restriction endonucleases are the enzymes that cleave DNA molecules at specific nucleotide sequences depending on the particular enzyme used. Enzyme recognition sites are usually 4 to 6 base pairs in length. Generally, the shorter the recognition sequence, the greater the number of fragments generated. If molecules differ in nucleotide sequence, fragments of different sizes may be generated. The fragments can be separated by gel electrophoresis. Restriction enzymes are isolated from a wide variety of bacterial genera and are thought to be part of the cell's defenses against invading bacterial viruses. Use of RFLP and restriction endonucleases in genetic marker analysis, such as SNP analysis, requires that the SNP affect cleavage of at least one restriction enzyme site.

Primer Extension is a technique in which the primer and no more than three NTPs may be combined with a polymerase and the target sequence, which serves as a template for amplification. By using fewer than all four NTPs, it is possible to omit one or more of the polymorphic nucleotides needed for incorporation at the polymorphic site. The amplification may be designed such that the omitted nucleotide(s) is(are) not required between the 3′ end of the primer and the target polymorphism. The primer is then extended by a nucleic acid polymerase, such as Taq polymerase. If the omitted NTP is required at the polymorphic site, the primer is extended up to the polymorphic site, at which point the polymerization ceases. However, if the omitted NTP is not required at the polymorphic site, the primer will be extended beyond the polymorphic site, creating a longer product. Detection of the extension products is based on, for example, separation by size/length which will thereby reveal which polymorphism is present.

Oligonucleotide Hybridization is a technique in which oligonucleotides may be designed to hybridize directly to a target site of interest. The hybridization can be performed on any useful format. For example, oligonucleotides may be arrayed on a chip or plate in a microarray. Microarrays comprise a plurality of oligos spatially distributed over, and stably associated with, the surface of a substantially planar substrate, e.g., a biochip. Microarrays of oligonucleotides have been developed and find use in a variety of applications, such as screening and DNA sequencing.

In gene analysis with microarrays, an array of “probe” oligonucleotides is contacted with a nucleic acid sample of interest, i.e., a target. Contact is carried out under hybridization conditions and unbound nucleic acid is then removed. The resultant pattern of hybridized nucleic acid provides information regarding the genetic profile of the sample tested. Methodologies of gene analysis on microarrays are capable of providing both qualitative and quantitative information.

A variety of different arrays which may be used is known in the art. The probe molecules of the arrays which are capable of sequence-specific hybridization with target nucleic acid may be polynucleotides or hybridizing analogues or mimetics thereof, including: nucleic acids in which the phosphodiester linkage has been replaced with a substitute linkage, such as phosphorothioate, methylimino, methylphosphonate, phosphoramidate, guanidine and the like; and nucleic acids in which the ribose subunit has been substituted, e.g., hexose phosphodiester, peptide nucleic acids, and the like. The length of the probes will generally range from 10 to 1000 nts, wherein in some embodiments the probes will be oligonucleotides and usually range from 15 to 150 nts and more usually from 15 to 100 nts in length, and in other embodiments the probes will be longer, usually ranging in length from 150 to 1000 nts, where the polynucleotide probes may be single- or double-stranded, usually single-stranded, and may be PCR fragments amplified from cDNA.

Probe molecules arrayed on the surface of a substrate may correspond to selected genes being analyzed and be positioned on the array at a known location so that positive hybridization events may be correlated to expression of a particular gene in the physiological source from which the target nucleic acid sample is derived. The substrate with which the probe molecules are stably associated may be fabricated from a variety of materials, including plastics, ceramics, metals, gels, membranes, glasses, and the like. The arrays may be produced according to any convenient methodology, such as preforming the probes and then stably associating them with the surface of the support or growing the probes directly on the support. Different array configurations and methods for their production and use are known to those of skill in the art and disclosed, for example, in U.S. Pat. Nos. 5,445,934, 5,532,128, 5,556,752, 5,242,974, 5,384,261, 5,405,783, 5,412,087, 5,424,186, 5,429,807, 5,436,327, 5,472,672, 5,527,681, 5,529,756, 5,545,531, 5,554,501, 5,561,071, 5,571,639, 5,593,839, 5,599,695, 5,624,711, 5,658,734, 5,700,637, and 6,004,755, the disclosures of which are herein incorporated by reference in their entireties.

Following hybridization, where non-hybridized labeled nucleic acid is capable of emitting a signal during the detection step, a washing step is employed in which unhybridized labeled nucleic acid is removed from the support surface, generating a pattern of hybridized nucleic acid on the substrate surface. Various wash solutions and protocols for their use are known to those of skill in the art and may be used.

Where the label on the target nucleic acid is not directly detectable, the array comprising bound target may be contacted with the other member(s) of the signal producing system that is being employed. For example, where the target is biotinylated, the array may be contacted with streptavidin-fluorescer conjugate under conditions sufficient for binding between the specific binding member pairs to occur. Following contact, any unbound members of the signal producing system will then be removed, e.g., by washing. The specific wash conditions employed will depend on the specific nature of the signal producing system that is employed, as will be known to those of skill in the art familiar with the particular signal producing system employed.

The resultant hybridization pattern(s) of labeled nucleic acids may be visualized or detected in a variety of ways, with the particular manner of detection being chosen based on the particular label of the nucleic acid, where representative detection means include scintillation counting, autoradiography, fluorescence measurement, calorimetric measurement, light emission measurement and the like.

Prior to detection or visualization, the potential for a mismatch hybridization event that could potentially generate a false positive signal on the pattern may be reduced by treating the array of hybridized target/probe complexes with an endonuclease under conditions sufficient such that the endonuclease degrades single stranded, but not double stranded, DNA. Various different endonucleases are known and may be used, including but not limited to mung bean nuclease, Si nuclease, and the like. Where such treatment is employed in an assay in which the target nucleic acids are not labeled with a directly detectable label, e.g., in an assay with biotinylated target nucleic acids, the endonuclease treatment will generally be performed prior to contact of the array with the other member(s) of the signal producing system, e.g., fluorescent-streptavidin conjugate. Endonuclease treatment, as described above, ensures that only end-labeled target/probe complexes having a substantially complete hybridization at the 3′ end of the probe are detected in the hybridization pattern.

Following hybridization and any washing step(s) and/or subsequent treatments, as described herein, the resultant hybridization pattern may be detected. In detecting or visualizing the hybridization pattern, the intensity or signal value of the label may also be quantified, such that the signal from each spot of the hybridization will be measured and compared to a unit value corresponding the signal emitted by known number of labeled target nucleic acids to obtain a count or absolute value of the copy number of each end-labeled target that is hybridized to a particular spot on the array in the hybridization pattern.

It will be appreciated that any useful system for detecting nucleic acids may be used in accordance with the present disclosure. For example, mass spectrometry, hybridization, sequencing, labeling, and separation analysis may be used individually or in combination, and may also be used in combination with other known methods of detecting nucleic acids.

Electrospray ionization (ESI) is a type of mass spectrometry that is used to produce gaseous ions from highly polar, mostly nonvolatile biomolecules, including lipids. The sample is typically injected as a liquid at low flow rates (1-10 μL/min) through a capillary tube to which a strong electric field is applied. The field charges the liquid in the capillary and produces a fine spray of highly charged droplets that are electrostatically attracted to the mass spectrometer inlet. The evaporation of the solvent from the surface of a droplet as it travels through the desolvation chamber increases its charge density substantially. When this increase exceeds the Rayleigh stability limit, ions are ejected and ready for MS analysis.

A typical conventional ESI source consists of a metal capillary of typically 0.1-0.3 mm in diameter, with a tip held approximately 0.5 to 5 cm (but more usually 1 to 3 cm) away from an electrically grounded circular interface having at its center the sampling orifice. A potential difference of between 1 to 5 kV (but more typically 2 to 3 kV) is applied to the capillary by power supply to generate a high electrostatic field (10⁶ to 10⁷ V/m) at the capillary tip. A sample liquid, carrying the analyte to be analyzed by the mass spectrometer, is delivered to the tip through an internal passage from a suitable source (such as from a chromatograph or directly from a sample solution via a liquid flow controller). By applying pressure to the sample in the capillary, the liquid leaves the capillary tip as small highly electrically charged droplets and further undergoes desolvation and breakdown to form single or multi-charged gas phase ions in the form of an ion beam. The ions are then collected by the grounded (or oppositely-charged) interface plate and led through an the orifice into an analyzer of the mass spectrometer. During this operation, the voltage applied to the capillary is held constant. Aspects of construction of ESI sources are described, for example, in U.S. Pat. Nos. 5,838,002; 5,788,166; 5,757,994; RE 35,413; and 5,986,258.

In ESI tandem mass spectroscopy (ESI/MS/MS), one is able to simultaneously analyze both precursor ions and product ions, thereby monitoring a single precursor product reaction and producing (through selective reaction monitoring (SRM)) a signal only when the desired precursor ion is present. When the internal standard is a stable isotope-labeled version of the analyte, this is known as quantification by the stable isotope dilution method. This approach has been used to accurately measure pharmaceuticals and bioactive peptides.

Secondary ion mass spectroscopy (SIMS) is an analytical method that uses ionized particles emitted from a surface for mass spectroscopy at a sensitivity of detection of a few parts per billion. The sample surface is bombarded by primary energetic particles, such as electrons, ions (e.g., O, Cs), neutrals or photons, forcing atomic and molecular particles to be ejected from the surface, a process called sputtering. Since some of these sputtered particles carry a charge, a mass spectrometer can be used to measure their mass and charge. Continued sputtering permits measuring of the exposed elements as material is removed. This in turn permits one to construct elemental depth profiles. Although the majority of secondary ionized particles are electrons, it is the secondary ions which are detected and analyzed by the mass spectrometer in this method.

Laser desorption mass spectroscopy (LD-MS) involves the use of a pulsed laser, which induces desorption of sample material from a sample site, and effectively, vaporizes sample off of the sample substrate. This method is usually used in conjunction with a mass spectrometer, and can be performed simultaneously with ionization by adjusting the laser radiation wavelength.

When coupled with Time-of-Flight (TOF) measurement, LD-MS is referred to as LDLPMS (Laser Desorption Laser Photoionization Mass Spectroscopy). The LDLPMS method of analysis gives instantaneous volatilization of the sample, and this form of sample fragmentation permits rapid analysis without any wet extraction chemistry. The LDLPMS instrumentation provides a profile of the species present while the retention time is low and the sample size is small. In LDLPMS, an impactor strip is loaded into a vacuum chamber. The pulsed laser is fired upon a certain spot of the sample site, and species present are desorbed and ionized by the laser radiation. This ionization also causes the molecules to break up into smaller fragment-ions. The positive or negative ions made are then accelerated into the flight tube, being detected at the end by a microchannel plate detector. Signal intensity, or peak height, is measured as a function of travel time. The applied voltage and charge of the particular ion determines the kinetic energy, and separation of fragments is due to their different sizes causing different velocities. Each ion mass will thus have a different flight-time to the detector.

Other advantages of the LDLPMS method include the possibility of constructing the system to give a quiet baseline of the spectra because one can prevent coevolved neutrals from entering the flight tube by operating the instrument in a linear mode. Also, in environmental analysis, the salts in the air and as deposits will not interfere with the laser desorption and ionization. This instrumentation also is very sensitive and robust, and has been shown to be capable of detecting trace levels in natural samples without any prior extraction preparations.

Matrix Assisted Laser Desorption/Ionization Time-of Flight (MALDI-TOF) is a type of mass spectrometry useful for analyzing molecules across an extensive mass range with high sensitivity, minimal sample preparation and rapid analysis times. MALDI-TOF also enables non-volatile and thermally labile molecules to be analyzed with relative ease. One important application of MALDI-TOF is in the area of quantification of peptides and proteins, such as in biological tissues and fluids.

Surface Enhanced Laser Desorption and Ionization (SELDI) is another type of desorption/ionization gas phase ion spectrometry in which an analyte is captured on the surface of a SELDI mass spectrometry probe. There are several known versions of SELDI.

One version of SELDI is affinity capture mass spectrometry, also called Surface-Enhanced Affinity Capture (SEAC). This version involves the use of probes that have a material on the probe surface that captures analytes through a non-covalent affinity interaction (adsorption) between the material and the analyte. The material is variously called an “adsorbent,” a “capture reagent,” an “affinity reagent” or a “binding moiety.” The capture reagent may be any material capable of binding an analyte. The capture reagent may be attached directly to the substrate of the selective surface, or the substrate may have a reactive surface that carries a reactive moiety that is capable of binding the capture reagent, e.g., through a reaction forming a covalent or coordinate covalent bond. Epoxide and carbodiimidizole are useful reactive moieties to covalently bind polypeptide capture reagents such as antibodies or cellular receptors. Nitriloacetic acid and iminodiacetic acid are useful reactive moieties that function as chelating agents to bind metal ions that interact non-covalently with histidine containing peptides. Adsorbents are generally classified as chromatographic adsorbents and biospecific adsorbents.

Another version of SELDI is Surface-Enhanced Neat Desorption (SEND), which involves the use of probes comprising energy absorbing molecules that are chemically bound to the probe surface. Energy absorbing molecules (EAM) refer to molecules that are capable of absorbing energy from a laser desorption/ionization source and, thereafter, of contributing to desorption and ionization of analyte molecules in contact therewith. The EAM category includes molecules used in MALDI, frequently referred to as “matrix,” and is exemplified by cinnamic acid derivatives such as sinapinic acid (SPA), cyano-hydroxy-cinnamic acid (CHCA) and dihydroxybenzoic acid, ferulic acid, and hydroxyaceto-phenone derivatives. In certain versions, the energy absorbing molecule is incorporated into a linear or cross-linked polymer, e.g., a polymethacrylate. For example, the composition may be a co-polymer of α-cyano-4-methacryloyloxycinnamic acid and acrylate. In another version, the composition may be a co-polymer of α-cyano-4-methacryloyloxycinnamic acid, acrylate and 3-(tri-ethoxy)silyl propyl methacrylate. In another version, the composition may be a co-polymer of α-cyano-4-methacryloyloxycinnamic acid and octadecylmethacrylate (“C18 SEND”).

SEAC/SEND is a version of SELDI in which both a capture reagent and an energy absorbing molecule are attached to the sample presenting surface. SEAC/SEND probes therefore allow the capture of analytes through affinity capture and ionization/desorption without the need to apply external matrix.

Another version of SELDI, called Surface-Enhanced Photolabile Attachment and Release (SEPAR), involves the use of probes having moieties attached to the surface that can covalently bind an analyte, and then release the analyte through breaking a photolabile bond in the moiety after exposure to light, e.g., to laser light. SEPAR and other forms of SELDI are readily adapted to detecting a marker or marker profile, in accordance with the present disclosure.

In accordance with the disclosure, nucleic acid hybridization is another useful method of analyzing genetic markers. Nucleic acid hybridization is generally understood as the ability of a nucleic acid to selectively form duplex molecules with complementary stretches of DNAs and/or RNAs. Depending on the application, varying conditions of hybridization may be used to achieve varying degrees of selectivity of the probe or primers for the target sequence.

Typically, a probe or primer of between 10 and 100 nucleotides, and up to 1-2 kilobases or more in length, will allow the formation of a duplex molecule that is both stable and selective. Molecules having complementary sequences over contiguous stretches greater than 20 bases in length may be used to increase stability and selectivity of the hybrid molecules obtained. Nucleic acid molecules for hybridization may be readily prepared, for example, by directly synthesizing the fragment by chemical means or by introducing selected sequences into recombinant vectors for recombinant production.

For applications requiring high selectivity, relatively high stringency conditions may be used to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, lower stringency conditions may be used. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated by those of skill depending on the desired results.

It is within the purview of the skilled artisan to design and select the appropriate primers, probes, and enzymes for any of the methods of genetic marker analysis. For example, for detection of SNPs, the skilled artisan will generally use agents that are capable of detecting single nucleotide changes in DNA. These agents may hybridize to target sequences that contain the change. Or, these agents may hybridize to target sequences that are adjacent to (e.g., upstream or 5′ to) the region of change.

In general, it is envisioned that the probes or primers described herein will be useful as reagents in solution hybridization for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest, as described herein, is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section are incorporated herein by reference.

The synthesis of oligonucleotides for use as primers and probes is well known to those of skill in the art. Chemical synthesis can be achieved, for example, by the diester method, the triester method, the polynucleotide phosphorylase method and by solid-phase chemistry. Various mechanisms of oligonucleotide synthesis have been disclosed, for example, in U.S. Pat. Nos. 4,659,774, 4,816,571, 5,141,813, 5,264,566, 4,959,463, 5,428,148, 5,554,744, 5,574,146, and 5,602,244, each of which is incorporated herein by reference in its entirety.

In certain embodiments, nucleic acid products are separated by agarose, agarose-acrylamide or polyacrylamide gel electrophoresis using standard methods such as those described, for example, in Sambrook et al., 1989. Separated products may be cut out and eluted from the gel for further manipulation. Using low melting point agarose gels, the skilled artisan may remove the separated band by heating the gel, followed by extraction of the nucleic acid.

Separation of nucleic acids may also be effected by chromatographic techniques known in the art. There are many kinds of chromatography that may be used in the practice of the present invention, non-limiting examples of which include capillary adsorption, partition, ion-exchange, hydroxylapatite, molecular sieve, reverse-phase, column, paper, thin-layer, and gas chromatography, as well as HPLC.

A number of the above separation platforms may be coupled to achieve separations based on two different properties. For example, some of the primers may be coupled with a moiety that allows affinity capture, and some primers remain unmodified. Modifications may include a sugar (for binding to a lectin column), a hydrophobic group (for binding to a reverse-phase column), biotin (for binding to a streptavidin column), or an antigen (for binding to an antibody column). Samples may be run through an affinity chromatography column. The flow-through fraction is collected, and the bound fraction eluted (by chemical cleavage, salt elution, etc.). Each sample may then be further fractionated based on a property, such as mass, to identify individual components.

In certain aspects, it will be advantageous to employ nucleic acids of defined sequences of the present disclosure in combination with an appropriate means, such as a label, for determining hybridization. Various appropriate indicator means are known in the art, including fluorescent, radioactive, enzymatic or other ligands, such as avidin/biotin, which are capable of being detected. In the case of enzyme tags, colorimetric indicator substrates are known that may be employed to provide a detection means that is visibly or spectrophotometrically detectable, to identify specific hybridization with complementary nucleic acid containing samples. In yet other embodiments, the primer has a mass label that can be used to detect the molecule amplified. Other embodiments also contemplate the use of Taqman™ and Molecular Beacon™ probes.

Radioactive isotopes useful for the practice of the invention include, but are not limited to, tritium, ¹⁴C and ³²P. Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

The choice of label may vary, depending on the method used for analysis. When using capillary electrophoresis, microfluidic electrophoresis, HPLC, or LC separations, either incorporated or intercalated fluorescent dyes may be used to label and detect the amplification products. Samples are detected dynamically, in that fluorescence is quantitated as a labeled species moves past the detector. If an electrophoretic method, HPLC, or LC is used for separation, products can be detected by absorption of UV light. If polyacrylamide gel or slab gel electrophoresis is used, the primer for the extension reaction can be labeled with a fluorophore, a chromophore or a radioisotope, or by associated enzymatic reaction. Alternatively, if polyacrylamide gel or slab gel electrophoresis is used, one or more of the NTPs in the extension reaction can be labeled with a fluorophore, a chromophore or a radioisotope, or by associated enzymatic reaction. Enzymatic detection involves binding an enzyme to a nucleic acid, e.g., via a biotin:avidin interaction, following separation of the amplification products on a gel, then detection by chemical reaction, such as chemiluminescence generated with luminol. A fluorescent signal may be monitored dynamically. Detection with a radioisotope or enzymatic reaction may require an initial separation by gel electrophoresis, followed by transfer of DNA molecules to a solid support (blot) prior to analysis. If blots are made, they can be analyzed more than once by probing, stripping the blot, and then reprobing. If the extension products are separated using a mass spectrometer, no label is required because nucleic acids are detected directly.

While whole genome association (WGA) studies allow examination of many common SNPs in different individuals to identify associations between SNPs and traits like major diseases, exome sequencing studies can increase efficiency by allowing selective sequencing of at least the coding regions (i.e., the exons that are translated into proteins) of the genome, in which most functional variation is thought to occur. Some benefits of exome sequencing can include the detection of traits without traditional genetic linkage, with fewer available case studies (e.g., rare Mendelian diseases), with causal variants in different genes (i.e., genetic heterogeneity), and with diverse clinical features (i.e., phenotypic heterogeneity). The exome constitutes only about 1% of the entire human genome, and a large number of rare mutations have weak or no effects in non-coding sequences.

Target-enrichment methods like direct genomic selection (DGS) allow selective capture of genomic regions of interest from a DNA sample prior to sequencing. Other target-enrichment methods can include, but are not limited to, at least one of polymerase chain reaction (PCR) to amplify target-specific DNA sequences; molecular inversion probes of single-stranded DNA oligonucleotides that undergo an enzymatic reaction with target-specific DNA sequences to form circular DNA fragments; hybrid capture microarrays that contain fixed, tiled single-stranded DNA oligonucleotides with target-specific DNA sequences to hybridize sheared double-stranded fragments of genomic DNA; in-solution capture with single-stranded DNA oligonucleotides with target-specific DNA sequences synthesized in solution to hybridize sheared double-stranded fragments of genomic DNA in the solution; and methods using sequencing platforms, such as Sanger sequencing, 454™ sequencing (available from Roche Diagnostics Corp. (Branford, Conn.)), the Genome Analyzer™ (available from Illumina, Inc. (San Diego, Calif.)), and SOLiD® and Ion Torrent™ technologies (available from Life Technologies Corp. (Carlsbad, Calif.)).

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference in its entirety.

While the foregoing specification teaches the principles of the invention, with examples provided for the purpose of illustration, it will be appreciated by one skilled in the art from reading this disclosure that various changes in form and detail can be made without departing from the true scope of the invention.

### EXAMPLES

### Example 1

### Whole-Genome Association Study

A whole-genome association (WGA) study was undertaken in which the case group comprised 358 cases (177 spontaneous ONJ cases and 181 surgically-induced ONJ cases). ONJ cases were characterized using comprehensive clinical report formats.

The control group comprised 2023 samples that match the cases for age, sex, and race. The controls were categorized as penicillin negative, POPRES, TSI, ALS, and Hypergenes Italian subject cohorts.

Genotyping was performed using the Illumina Human1M BeadChip platform, which contains 1072820 probes for SNPs and Copy Number Variations (CNVs). Genotyping was also performed using the Illumina 1M-Duo and Illumina 550K chips.

Principle component analysis (PCA) was done on all ONJ cases and controls to detect population structure. Standard quality control procedures were applied to the case-control genotype data set (based on SNP call rates, Hardy-Weinberg Equilibrium, and minor allele frequency) to exclude from downstream analysis low quality SNPs that could generate potentially false positive associations. Genetically-matched controls were selected for each case group, resulting in 358 cases (177 spontaneous ONJ cases and 181 surgically-induced ONJ cases).

Associations were tested using Fisher's exact test under additive, dominant, and recessive models through PLINK. The cohorts analyzed against the 2023 controls in the WGA study were: total ONJ cases, spontaneous ONJ cases, surgically-induced ONJ cases, and drug-specific analyses of zolendronate and alendronate cases. Tables 1-5 show the SNPs that have a p-value smaller than 10⁻⁵ in each of the data sets.

Total ONJ Cases vs. Controls

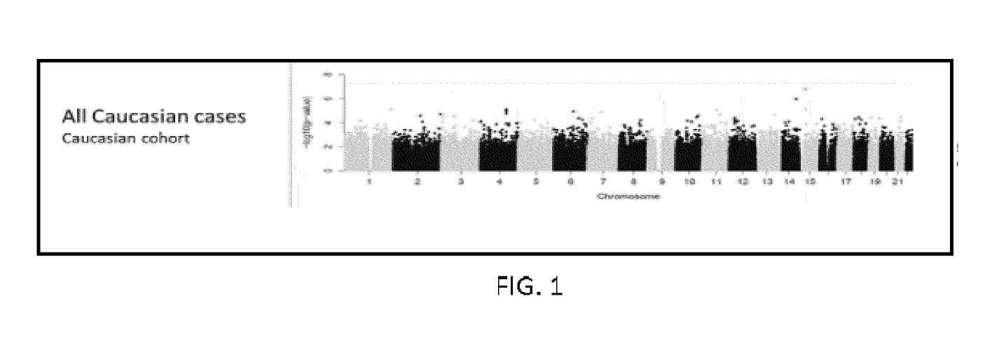

Table 1 shows the SNPs found to be the most strongly associated with ONJ. FIG. 1 is a Manhattan plot summarizing the results of the WGA study for all cases.

TABLE 1

| | | Position (NCBI | | |
|---|---|---|---|---|
| SNP Name | Chromosome | Build 37) | p-value | Odds Ratio |
| rs12440268 | 15 | 30200346 | 1.63E−07 | 2.163 |
| rs10484024 | 14 | 89834382 | 1.08E−06 | 0.6618 |
| rs785425 | 15 | 30178614 | 6.91E−06 | 1.777 |
| rs2253244 | 1 | 237928320 | 7.35E−06 | 1.467 |
| rs995637 | 4 | 130364383 | 7.48E−06 | 1.446 |
| rs4340077 | 11 | 70890503 | 7.86E−06 | 1.627 |
| rs1433375 | 4 | 130338059 | 1.10E−05 | 1.435 |
| rs595074 | 6 | 98240442 | 1.14E−05 | 1.476 |
| rs11971450 | 7 | 67084772 | 1.27E−05 | 1.654 |
| rs1816429 | 4 | 130347005 | 1.64E−05 | 1.424 |
| rs1400671 | 3 | 5992687 | 1.88E−05 | 1.433 |
| rs7597880 | 2 | 237958171 | 1.95E−05 | 1.721 |
| rs595739 | 11 | 88014239 | 1.99E−05 | 1.619 |
| rs540202 | 13 | 93710618 | 2.26E−05 | 1.493 |
| rs6759065 | 2 | 135389197 | 2.68E−05 | 1.409 |
| rs7916268 | 10 | 113295975 | 2.78E−05 | 1.476 |
| rs1517931 | 3 | 65018403 | 3.04E−05 | 0.681 |
| rs2836469 | 21 | 39897553 | 3.27E−05 | 1.404 |
| rs12772784 | 10 | 105466808 | 3.50E−05 | 1.549 |
| rs10502510 | 18 | 26225616 | 3.70E−05 | 0.607 |
| rs4804078 | 19 | 8787140 | 3.75E−05 | 0.6648 |
| rs9810413 | 3 | 5962755 | 3.86E−05 | 1.413 |
| rs7315570 | 12 | 27061770 | 3.96E−05 | 1.481 |
| rs4806495 | 19 | 54587396 | 4.20E−05 | 0.4417 |
| rs886791 | 7 | 23330347 | 4.36E−05 | 0.6913 |
| rs12200663 | 6 | 125434358 | 4.43E−05 | 1.406 |
| rs12598337 | 16 | 10440804 | 4.81E−05 | 1.58 |
| rs8094017 | 18 | 8882679 | 4.97E−05 | 0.7108 |
| rs8089412 | 18 | 38451425 | 5.49E−05 | 1.539 |
| rs10514228 | 5 | 80924474 | 5.57E−05 | 2.511 |
| rs11754065 | 6 | 143621631 | 5.82E−05 | 1.538 |
| rs3134127 | 8 | 107709845 | 5.99E−05 | 1.641 |
| rs10842812 | 12 | 27036420 | 6.06E−05 | 1.466 |
| rs6792827 | 3 | 158545195 | 6.57E−05 | 1.506 |
| rs10785342 | 12 | 42895401 | 6.96E−05 | 0.708 |
| rs4275536 | 10 | 59181959 | 7.08E−05 | 2.055 |
| rs2836480 | 21 | 39907437 | 7.47E−05 | 1.385 |
| rs575528 | 1 | 81563249 | 7.51E−05 | 0.7106 |
| rs1426782 | 1 | 81516862 | 7.58E−05 | 0.7064 |
| rs708158 | 12 | 27087703 | 7.90E−05 | 1.457 |
| rs3817604 | 4 | 1291337 | 7.91E−05 | 1.602 |
| rs1776503 | 14 | 52687869 | 8.20E−05 | 1.383 |
| rs1532451 | 12 | 42141172 | 8.20E−05 | 1.454 |
| rs12218276 | 10 | 80271486 | 8.21E−05 | 1.544 |
| rs28429103 | 4 | 1320023 | 8.27E−05 | 1.593 |
| rs13026163 | 2 | 145866511 | 8.37E−05 | 1.795 |
| rs10824620 | 10 | 80266999 | 8.67E−05 | 1.538 |
| rs2048923 | 3 | 6010772 | 9.24E−05 | 1.429 |
| rs9325124 | 5 | 148248818 | 9.42E−05 | 0.7173 |
| rs11156787 | 14 | 33841444 | 9.47E−05 | 1.376 |
| rs4936509 | 11 | 120041444 | 9.56E−05 | 0.7128 |

Spontaneous ONJ Cases vs. Controls

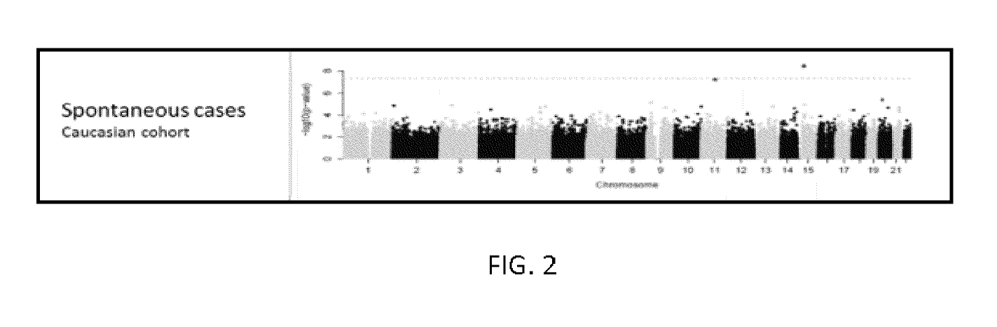

Table 2 shows the SNPs found to be the most strongly associated with spontaneous ONJ. FIG. 2 is a Manhattan plot summarizing the results of the WGA study for spontaneous ONJ cases.

TABLE 2

| | | Position (NCBI | | |
|---|---|---|---|---|
| SNP Name | Chromosome | Build 37) | p-value | Odds Ratio |
| rs12440268 | 15 | 30200346 | 3.69E−09 | 2.92 |
| rs4340077 | 11 | 70890503 | 6.46E−08 | 2.14 |
| rs6045177 | 20 | 18048084 | 4.15E−06 | 0.4585 |
| rs17692260 | 9 | 21094833 | 7.12E−06 | 2.317 |
| rs785425 | 15 | 30178614 | 1.24E−05 | 2.05 |
| rs308022 | 2 | 6873356 | 1.38E−05 | 0.2059 |
| rs10212434 | 3 | 56432160 | 1.48E−05 | 0.5853 |
| rs308027 | 2 | 6874452 | 1.67E−05 | 0.2091 |
| rs540202 | 13 | 93710618 | 1.71E−05 | 1.725 |
| rs318373 | 5 | 143319871 | 1.72E−05 | 0.6021 |
| rs10857733 | 10 | 135332803 | 1.72E−05 | 2.305 |
| rs2836489 | 21 | 39910680 | 1.95E−05 | 0.5815 |
| rs4266447 | 5 | 116406053 | 2.17E−05 | 1.833 |
| rs2836480 | 21 | 39907437 | 2.19E−05 | 1.62 |
| rs11086190 | 20 | 45671827 | 2.32E−05 | 1.912 |
| rs7868651 | 9 | 95555939 | 2.33E−05 | 0.5972 |
| rs3783853 | 14 | 89803269 | 2.59E−05 | 1.621 |
| rs2836469 | 21 | 39897553 | 3.39E−05 | 1.592 |
| rs13124016 | 4 | 57167280 | 3.45E−05 | 1.905 |
| rs8094017 | 18 | 8882679 | 3.64E−05 | 0.6156 |
| rs4890055 | 17 | 78587225 | 3.81E−05 | 1.86 |
| rs563 | 9 | 139296485 | 4.11E−05 | 1.681 |
| rs6595008 | 5 | 116421862 | 5.21E−05 | 1.692 |
| rs2273704 | 14 | 100384232 | 5.55E−05 | 1.573 |
| rs506382 | 21 | 40977826 | 5.71E−05 | 1.607 |
| rs17670378 | 7 | 67975794 | 6.10E−05 | 2.459 |
| rs6794065 | 3 | 191256891 | 6.36E−05 | 1.586 |
| rs7733755 | 5 | 116416022 | 6.37E−05 | 1.79 |
| rs10441708 | 9 | 23141807 | 7.00E−05 | 2.164 |
| rs1340978 | 1 | 161676970 | 7.16E−05 | 1.558 |
| rs703704 | 12 | 101273368 | 7.43E−05 | 1.677 |
| rs10767741 | 11 | 28700423 | 7.50E−05 | 0.6079 |
| rs10484024 | 14 | 89834382 | 7.81E−05 | 0.6311 |
| rs10875295 | 1 | 100970346 | 7.95E−05 | 0.6382 |
| rs10787675 | 10 | 118237852 | 8.08E−05 | 1.971 |
| rs3763046 | 19 | 5823903 | 8.10E−05 | 2.117 |
| rs1554074 | 12 | 101240350 | 9.00E−05 | 1.673 |
| rs10277926 | 7 | 33629413 | 9.10E−05 | 2.772 |
| rs8028396 | 15 | 32396721 | 9.48E−05 | 1.555 |

Surgically-Induced ONJ Cases vs. Controls

Table 3 shows the SNPs found to be the most strongly associated with surgically-induced ONJ.

TABLE 3

| | | Position (NCBI | | |
|---|---|---|---|---|
| SNP Name | Chromosome | Build 37) | p-value | Odds Ratio |
| rs7225045 | 17 | 6203690 | 1.29E−06 | 1.755 |
| rs6554641 | 5 | 11801562 | 3.72E−06 | 3.345 |
| rs7731468 | 5 | 11831211 | 5.10E−06 | 3.235 |
| rs1841217 | 7 | 16704191 | 8.12E−06 | 1.777 |
| rs2028028 | 7 | 112318992 | 8.16E−06 | 1.665 |
| rs10868546 | 9 | 89872124 | 1.04E−05 | 1.639 |
| rs4812586 | 20 | 35544673 | 1.09E−05 | 1.834 |
| rs3924181 | 5 | 11822833 | 1.18E−05 | 3.16 |
| rs6867416 | 5 | 11775395 | 1.59E−05 | 3.143 |
| rs2748663 | 20 | 37383640 | 1.69E−05 | 0.5369 |
| rs2757521 | 20 | 37383841 | 1.92E−05 | 0.539 |
| rs1969763 | 9 | 89874893 | 1.94E−05 | 1.611 |
| rs7487222 | 12 | 129936385 | 2.66E−05 | 1.611 |
| rs2243520 | 20 | 37395112 | 2.70E−05 | 0.5442 |
| rs3212254 | 14 | 24805463 | 3.16E−05 | 2.147 |
| rs9894371 | 17 | 6212579 | 3.42E−05 | 1.6 |
| rs12218276 | 10 | 80271486 | 3.53E−05 | 1.796 |
| rs2420963 | 10 | 123540904 | 3.79E−05 | 0.5927 |
| rs16852239 | 4 | 40477729 | 4.71E−05 | 1.64 |
| rs208896 | 21 | 18815516 | 4.79E−05 | 1.576 |
| rs3782067 | 11 | 2935303 | 5.07E−05 | 1.56 |
| rs225206 | 17 | 30894372 | 5.84E−05 | 0.6176 |
| rs208894 | 21 | 18814568 | 5.89E−05 | 1.564 |
| rs10498318 | 14 | 34179367 | 6.11E−05 | 1.574 |
| rs10082466 | 10 | 54526622 | 6.15E−05 | 1.601 |
| rs7558032 | 2 | 155026455 | 6.47E−05 | 2.549 |
| rs1401518 | 2 | 154800999 | 6.64E−05 | 2.096 |
| rs2253244 | 1 | 237928320 | 7.11E−05 | 1.566 |
| rs6741566 | 2 | 154772373 | 7.70E−05 | 2.082 |
| rs225209 | 17 | 30894886 | 7.79E−05 | 0.6183 |
| rs7186606 | 16 | 86929329 | 7.86E−05 | 1.781 |
| rs16918472 | 11 | 92801894 | 8.42E−05 | 1.803 |
| rs10824787 | 10 | 54507871 | 9.05E−05 | 1.598 |
| rs1364958 | 8 | 57540239 | 9.21E−05 | 1.606 |
| rs7984252 | 13 | 39703461 | 9.29E−05 | 1.838 |
| rs4697158 | 4 | 18841427 | 9.30E−05 | 0.6222 |
| rs8076681 | 17 | 6247468 | 9.65E−05 | 1.563 |
| rs732211 | 11 | 123485444 | 9.79E−05 | 0.6206 |

### Drug-Specific Analyses

For the zolendronate-specific analysis, a subset of 219 cases, comprising subjects who were treated with zolendronate, was analyzed. Table 4 shows the SNPs found to be the most strongly associated with zolendronate-induced ONJ.

TABLE 4

| | | Position (NCBI | | |
|---|---|---|---|---|
| SNP Name | Chromosome | Build 37) | p-value | Odds Ratio |
| rs12440268 | 15 | 30200346 | 2.24E−06 | 2.311 |
| rs2276424 | 11 | 118209960 | 4.28E−06 | 1.629 |
| rs4340077 | 11 | 70890503 | 7.43E−06 | 1.804 |
| rs7937334 | 11 | 118191274 | 9.65E−06 | 1.612 |
| rs4343763 | 4 | 186801590 | 1.27E−05 | 0.5791 |
| rs995637 | 4 | 130364383 | 1.44E−05 | 1.558 |
| rs11971450 | 7 | 67084772 | 1.60E−05 | 1.814 |
| rs12772784 | 10 | 105466808 | 1.68E−05 | 1.725 |
| rs7558032 | 2 | 155026455 | 2.08E−05 | 2.548 |
| rs643120 | 11 | 132390291 | 2.22E−05 | 1.727 |
| rs1433375 | 4 | 130338059 | 2.24E−05 | 1.54 |
| rs10931862 | 2 | 154830754 | 2.61E−05 | 2.479 |
| rs1816429 | 4 | 130347005 | 3.09E−05 | 1.527 |
| rs16918472 | 11 | 92801894 | 3.13E−05 | 1.785 |
| rs6049754 | 20 | 24504850 | 3.13E−05 | 2.019 |
| rs17014760 | 4 | 130340880 | 3.46E−05 | 1.58 |
| rs1999487 | 1 | 108408983 | 3.49E−05 | 0.6128 |
| rs7916268 | 10 | 113295975 | 3.55E−05 | 1.597 |
| rs10490537 | 2 | 154912289 | 3.58E−05 | 2.472 |
| rs4690060 | 4 | 2760222 | 3.78E−05 | 1.537 |
| rs7162940 | 15 | 100136381 | 3.84E−05 | 1.531 |
| rs9931159 | 16 | 22931029 | 3.99E−05 | 1.919 |
| rs13158321 | 5 | 141128076 | 4.13E−05 | 1.877 |
| rs9463046 | 6 | 44802718 | 4.14E−05 | 2.32 |
| rs11077904 | 17 | 75366580 | 4.85E−05 | 1.651 |
| rs10484024 | 14 | 89834382 | 5.37E−05 | 0.6541 |
| rs2131918 | 9 | 95353785 | 5.52E−05 | 0.6317 |
| rs16936474 | 9 | 116139835 | 5.66E−05 | 1.702 |
| rs2292750 | 17 | 40811781 | 5.82E−05 | 1.511 |
| rs13115937 | 4 | 26512016 | 6.28E−05 | 0.619 |
| rs753403 | 17 | 75366240 | 6.43E−05 | 1.64 |
| rs11640737 | 16 | 85795272 | 7.05E−05 | 1.875 |
| rs11787444 | 8 | 61294126 | 7.96E−05 | 1.617 |
| rs7868651 | 9 | 95555939 | 8.88E−05 | 0.6528 |
| rs9465995 | 6 | 21202154 | 8.91E−05 | 1.987 |
| rs17565061 | 4 | 108906300 | 9.57E−05 | 0.405 |
| rs1006703 | 17 | 3865399 | 9.61E−05 | 0.5286 |
| rs11648894 | 16 | 88766359 | 9.65E−05 | 1.517 |
| rs246636 | 5 | 142411430 | 9.90E−05 | 0.4707 |

For the alendronate-specific analysis, a subset of 94 cases, comprising subjects who were treated with alendronate, was analyzed. Table 5 shows the SNP found to be the most strongly associated with alendronate-induced ONJ.

TABLE 5

| | | Position (NCBI | | |
|---|---|---|---|---|
| SNP Name | Chromosome | Build 37) | p-value | Odds Ratio |
| rs2122268 | 12 | 26945413 | 1.08E−06 | 3.025 |
| rs7540743 | 1 | 61945181 | 3.58E−06 | 2.198 |
| rs1859416 | 7 | 8899901 | 5.21E−06 | 2.049 |
| rs4804078 | 19 | 8787140 | 8.02E−06 | 0.3637 |
| rs7301331 | 12 | 27355213 | 1.07E−05 | 2.207 |
| rs16999051 | 20 | 17361482 | 1.19E−05 | 2.327 |
| rs11086000 | 19 | 8809399 | 2.06E−05 | 0.4477 |
| rs2223271 | 22 | 34920388 | 2.36E−05 | 1.886 |
| rs9373821 | 6 | 106406999 | 2.56E−05 | 1.944 |
| rs16881815 | 4 | 29425637 | 2.63E−05 | 2.507 |
| rs6678876 | 1 | 182759592 | 2.86E−05 | 2.139 |
| rs2190218 | 7 | 81307464 | 3.30E−05 | 1.972 |
| rs2474366 | 1 | 61931141 | 3.34E−05 | 2.323 |
| rs6123724 | 20 | 56322729 | 3.87E−05 | 1.989 |
| rs12550790 | 8 | 110659239 | 4.72E−05 | 2.566 |
| rs16881347 | 5 | 52645739 | 4.78E−05 | 3.132 |
| rs9894506 | 17 | 11192281 | 4.81E−05 | 1.866 |
| rs1112008 | 11 | 69566488 | 5.35E−05 | 2.064 |
| rs1776503 | 14 | 52687869 | 5.72E−05 | 1.841 |
| rs7520347 | 1 | 241440418 | 5.77E−05 | 2.241 |
| rs841718 | 12 | 57492996 | 5.87E−05 | 0.5017 |
| rs2112824 | 19 | 35014298 | 6.18E−05 | 1.833 |
| rs6468694 | 8 | 100865836 | 6.19E−05 | 2.069 |
| rs12106074 | 20 | 17356566 | 6.45E−05 | 1.922 |
| rs1807143 | 2 | 76187099 | 6.47E−05 | 1.874 |
| rs4449697 | 7 | 18094115 | 6.61E−05 | 1.866 |
| rs2815854 | 1 | 241395811 | 6.75E−05 | 0.5099 |
| rs2815834 | 1 | 241401288 | 6.84E−05 | 1.863 |
| rs11185517 | 3 | 195931583 | 7.23E−05 | 1.931 |
| rs13047849 | 21 | 42421994 | 7.27E−05 | 1.85 |
| rs596647 | 11 | 119367321 | 7.64E−05 | 1.924 |
| rs7540172 | 1 | 61941583 | 7.70E−05 | 2.007 |
| rs1372381 | 5 | 162258547 | 7.71E−05 | 1.819 |
| rs4877836 | 9 | 86917088 | 7.73E−05 | 2.034 |
| rs214737 | 19 | 8813050 | 7.91E−05 | 0.517 |
| rs12050283 | 14 | 92286262 | 8.48E−05 | 2.223 |
| rs11152943 | 6 | 106405102 | 8.56E−05 | 1.854 |
| rs8184463 | 20 | 56339182 | 8.79E−05 | 1.898 |
| rs10882959 | 10 | 99304801 | 9.21E−05 | 1.892 |
| rs13266463 | 8 | 143403693 | 9.25E−05 | 1.814 |
| rs4853238 | 2 | 76578575 | 9.38E−05 | 1.838 |
| rs17504372 | 18 | 50903050 | 9.43E−05 | 1.81 |
| rs4877835 | 9 | 86916851 | 9.92E−05 | 2.014 |
| rs3751673 | 16 | 88552370 | 9.93E−05 | 1.806 |
| rs17123173 | 14 | 51387658 | 9.93E−05 | 3.7 |
| rs1037693 | 8 | 119028915 | 9.95E−05 | 1.792 |

### Example 2

### Whole-Genome Association Study

A whole-genome association (WGA) study was undertaken in which the case group comprised 358 cases. ONJ cases were characterized using comprehensive clinical report formats.

The control group comprised 2554 samples. The controls were categorized as penicillin negative, POPRES, TSI, WTCCC, Hypergenes Italian, Swedish and dbGaP Spanish cohorts.

Case and control cohorts were genotyped by different platforms at different time. Genotyping was performed using the Illumina Human1M Duo BeadChip platform or Human Core Exome Chip or by Human Omnia Express chip. The genotyping platforms contain different number of probes for SNPs and Copy Number Variations (CNVs). In order to increase the number of genetic variants, common across platforms, to be tested in the association analysis, untyped common genetic variants were predicted (imputed) for each sample based on the haplotype structure on the genome. In particular, samples were combined by genotyping chip, the data was phased using (Shapeit software) and, then, the genetic variants were imputed by IMPUTE v3 software using 1KG as reference library. Only the common SNPs (Minor allele frequency in general population greater than 1%), which were well-imputed (info greater than 0.4) in at least 95% of the samples, were then included in the final dataset tested for association.

Principle component analysis (PCA) was done on all ONJ cases and controls to detect population structure. Standard quality control procedures were applied to the case-control genotype data set (based on SNP call rates, Hardy-Weinberg Equilibrium, and minor allele frequency) to exclude from downstream analysis low quality SNPs that could generate potentially false positive associations. Genetically-matched controls were selected for each case group, resulting in 358 cases.

Associations were tested using Logistic regression test under additive, dominant, and recessive models through PLINK. The cohorts analyzed against the 2554 controls in the WGA study were: total ONJ cases, spontaneous ONJ cases and drug-specific analyses of zolendronate and alendronate cases. Two extreme phenotypes were also tested: the first phenotype (called Dimension) identifies as case any patient with a necrosis larger than 2.5 cm and the second (called Latency) identifies as case any patient with a onset shorter than 21 months from the treatment starting date. Tables 6-11 show the SNPs that have a p-value smaller than 10⁻⁶ in each of the data sets.

Total ONJ Cases vs. Controls

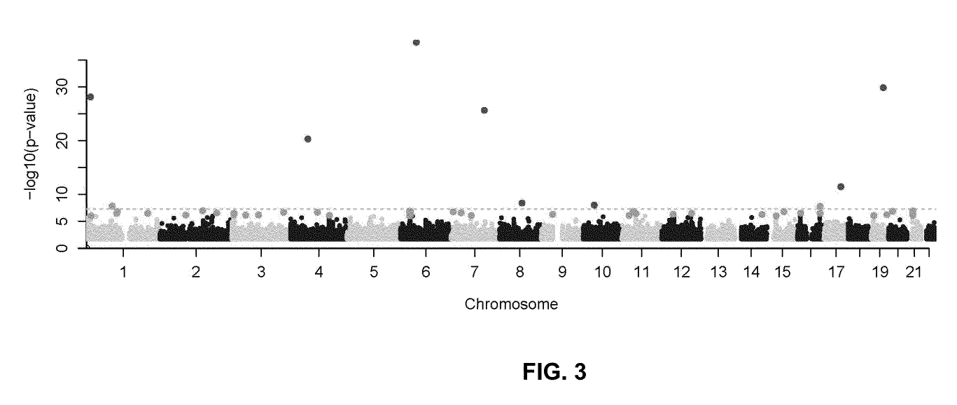

Table 6 shows the SNPs found to be the most strongly associated with ONJ. FIG. 3 is a Manhattan plot summarizing the results of the WGA study for all cases.

TABLE 6

| | | Position (NCBI | | |
|---|---|---|---|---|
| SNP Name | Chromosome | Build 37) | p-value | Odds Ratio |
| rs10874639 | 1 | 103133909 | 1.67E−07 | 1.584 |
| rs12093888 | 1 | 14654587 | 9.57E−07 | 2.834 |
| rs17346571 | 1 | 12835168 | 8.00E−29 | 5.014 |
| rs3768235 | 1 | 85733374 | 1.50E−08 | 2.291 |
| rs4908086 | 1 | 101088124 | 3.38E−07 | 0.64 |
| rs823136 | 1 | 205738251 | 3.43E−07 | 1.817 |
| rs5743130 | 2 | 190717628 | 2.83E−07 | 1.963 |
| rs7564795 | 2 | 143033292 | 1.09E−07 | 0.5662 |
| rs7579946 | 2 | 86043673 | 6.91E−07 | 1.487 |
| rs2323564 | 3 | 6011200 | 5.97E−07 | 1.529 |
| rs4686006 | 3 | 6018667 | 4.97E−07 | 1.525 |
| rs903894 | 3 | 89051475 | 6.79E−07 | 0.6324 |
| rs9846194 | 3 | 45683681 | 7.31E−07 | 0.02966 |
| rs9877241 | 3 | 173819047 | 2.19E−07 | 0.4649 |
| rs991619 | 3 | 6019957 | 3.26E−07 | 1.538 |
| rs13111701 | 4 | 90212077 | 2.11E−07 | 2.956 |
| rs1836066 | 4 | 130333906 | 7.98E−07 | 1.498 |
| rs2228991 | 4 | 57796900 | 5.11E−21 | 4.74 |
| rs112430285 | 6 | 32583880 | 8.22E−07 | 1.939 |
| rs114284967 | 6 | 32595682 | 8.23E−07 | 2.256 |
| rs116178292 | 6 | 29990937 | 9.68E−07 | 1.725 |
| rs116582397 | 6 | 29758525 | 1.35E−07 | 1.557 |
| rs116665189 | 6 | 32720971 | 1.80E−07 | 0.17 |
| rs147773060 | 6 | 32584894 | 7.33E−07 | 1.718 |
| rs2397118 | 6 | 52701143 | 5.75E−39 | 6.287 |
| rs59295208 | 6 | 32622229 | 1.43E−07 | 2.186 |
| rs11971450 | 7 | 67084772 | 8.68E−07 | 1.75 |
| rs17136102 | 7 | 6287840 | 1.73E−07 | 2.611 |
| rs2278819 | 7 | 32961524 | 2.59E−07 | 0.4638 |
| rs3757645 | 7 | 111426682 | 2.46E−26 | 5.22 |
| rs7795106 | 7 | 67080586 | 8.88E−07 | 1.752 |
| rs2717537 | 8 | 79699029 | 4.02E−09 | 1.768 |
| rs16933812 | 9 | 36969205 | 5.19E−07 | 0.635 |
| rs11010969 | 10 | 37264042 | 9.88E−09 | 1.859 |
| rs1435158 | 11 | 38468689 | 1.58E−07 | 1.591 |
| rs1822339 | 11 | 24359362 | 8.39E−07 | 0.6511 |
| rs7123576 | 11 | 47625046 | 3.82E−07 | 2.344 |
| rs11610034 | 12 | 100555590 | 3.29E−07 | 0.1204 |
| rs73097853 | 12 | 38103332 | 4.90E−07 | 2.336 |
| rs10484024 | 14 | 89834382 | 5.73E−07 | 0.6602 |
| rs2414451 | 15 | 56172652 | 1.67E−07 | 1.816 |
| rs56658700 | 15 | 30200027 | 9.96E−07 | 2.006 |
| rs117798852 | 16 | 75894335 | 3.30E−07 | 3.775 |
| rs142106800 | 16 | 75969590 | 1.71E−08 | 4.022 |
| rs8045362 | 16 | 10436543 | 2.96E−07 | 1.788 |
| rs12602205 | 17 | 56232675 | 3.83E−12 | 2.217 |
| rs117389731 | 19 | 40540697 | 1.53E−30 | 12 |
| rs76793736 | 19 | 8316252 | 8.36E−07 | 2.509 |
| rs78096315 | 19 | 8311734 | 9.30E−07 | 2.498 |
| rs8100716 | 19 | 52384061 | 5.81E−07 | 0.4449 |
| rs77255807 | 20 | 13251316 | 1.45E−07 | 2.695 |
| rs1038895 | 21 | 18776656 | 7.95E−07 | 2.244 |
| rs117935592 | 21 | 18782630 | 1.91E−07 | 2.329 |
| rs76372492 | 21 | 18778847 | 1.17E−07 | 2.396 |
| rs77559520 | 21 | 18770416 | 6.76E−07 | 2.386 |
| rs77914724 | 21 | 18782920 | 1.91E−07 | 2.329 |
| rs79134299 | 21 | 18785880 | 6.00E−07 | 2.27 |

Spontaneous ONJ Cases vs. Controls

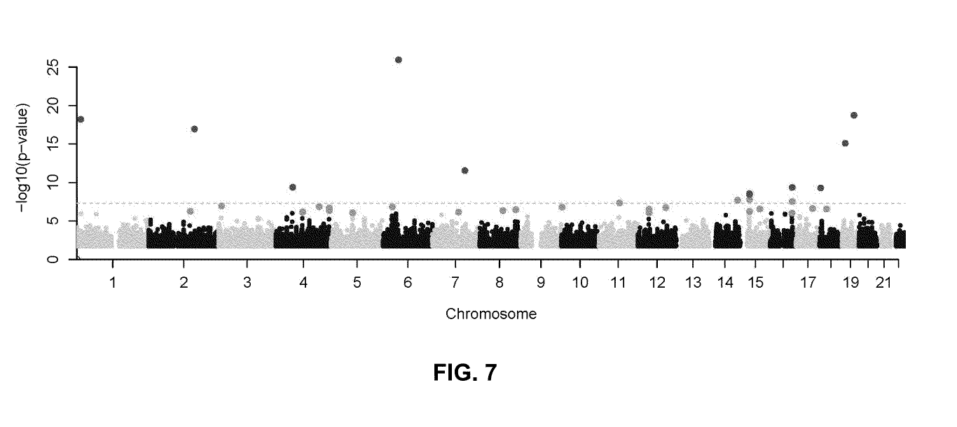

Table 7 shows the SNPs found to be the most strongly associated with spontaneous ONJ. FIG. 7 is a Manhattan plot summarizing the results of the WGA study for spontaneous ONJ cases.

TABLE 7

| | | Position (NCBI | | |
|---|---|---|---|---|
| SNP Name | Chromosome | Build 37) | p-value | Odds Ratio |
| rs17346571 | 1 | 12835168 | 6.51E−19 | 5.241 |
| rs7564795 | 2 | 143033292 | 5.79E−07 | 0.4388 |
| rs78750384 | 2 | 157854428 | 1.16E−17 | 4.75 |
| rs17050220 | 3 | 9350167 | 1.20E−07 | 2.043 |
| rs112677405 | 4 | 184466442 | 2.72E−07 | 2.425 |
| rs114145836 | 4 | 92058148 | 6.79E−07 | 4.142 |
| rs114694736 | 4 | 92113737 | 7.79E−07 | 4.105 |
| rs11723408 | 4 | 184462492 | 4.28E−07 | 2.441 |
| rs11727947 | 4 | 184472052 | 2.08E−07 | 2.483 |
| rs11729383 | 4 | 184467864 | 2.43E−07 | 2.471 |
| rs11737631 | 4 | 184467976 | 2.43E−07 | 2.471 |
| rs17024762 | 4 | 149464531 | 1.45E−07 | 2.354 |
| rs2228991 | 4 | 57796900 | 4.36E−10 | 3.981 |
| rs4286580 | 4 | 184464355 | 2.67E−07 | 2.463 |
| rs4621479 | 4 | 184466165 | 3.96E−07 | 2.43 |
| rs55723210 | 4 | 184463440 | 4.68E−07 | 2.433 |
| rs56114082 | 4 | 184465771 | 3.96E−07 | 2.43 |
| rs56242642 | 4 | 184463280 | 4.68E−07 | 2.433 |
| rs72691506 | 4 | 184465139 | 3.54E−07 | 2.439 |
| rs72691507 | 4 | 184465282 | 3.54E−07 | 2.439 |
| rs72691511 | 4 | 184469849 | 2.43E−07 | 2.471 |
| rs7438782 | 4 | 184467506 | 2.43E−07 | 2.471 |
| rs4704199 | 5 | 74563780 | 8.92E−07 | 2.253 |
| rs116369462 | 6 | 31253866 | 1.54E−07 | 2.018 |
| rs2397118 | 6 | 52701143 | 1.20E−26 | 6.667 |
| rs13224231 | 7 | 89521932 | 7.45E−07 | 2.425 |
| rs3757645 | 7 | 111426682 | 2.93E−12 | 4.194 |
| rs7828796 | 8 | 84280532 | 4.80E−07 | 1.887 |
| rs7837354 | 8 | 128596883 | 3.58E−07 | 0.223 |
| rs4880975 | 10 | 2163119 | 1.63E−07 | 0.3178 |
| rs4340077 | 11 | 70890503 | 4.64E−08 | 2.128 |
| rs11543410 | 12 | 38219455 | 9.10E−07 | 2.459 |
| rs4002590 | 12 | 38132572 | 3.18E−07 | 2.732 |
| rs4762519 | 12 | 95885590 | 1.93E−07 | 0.347 |
| rs11628901 | 14 | 96010424 | 2.10E−08 | 2.078 |
| rs12440268 | 15 | 30200346 | 1.68E−08 | 2.634 |
| rs2572217 | 15 | 66062227 | 2.88E−07 | 1.975 |
| rs56658700 | 15 | 30200027 | 2.92E−09 | 2.783 |
| rs60419830 | 15 | 30200210 | 3.68E−09 | 2.753 |
| rs76215974 | 15 | 30209827 | 6.07E−07 | 2.619 |
| rs117798852 | 16 | 75894335 | 3.08E−08 | 5.48 |
| rs142106800 | 16 | 75969590 | 4.55E−10 | 5.942 |
| rs16957558 | 16 | 75269325 | 9.94E−07 | 3.64 |
| rs12602205 | 17 | 56232675 | 2.49E−07 | 2.2 |
| rs1791084 | 18 | 3339405 | 5.24E−10 | 2.37 |
| rs9965541 | 18 | 23436642 | 2.86E−07 | 0.221 |
| rs117389731 | 19 | 40540697 | 1.89E−19 | 10.82 |
| rs2304255 | 19 | 10475649 | 8.33E−16 | 3.224 |

### Drug-Specific Analyses

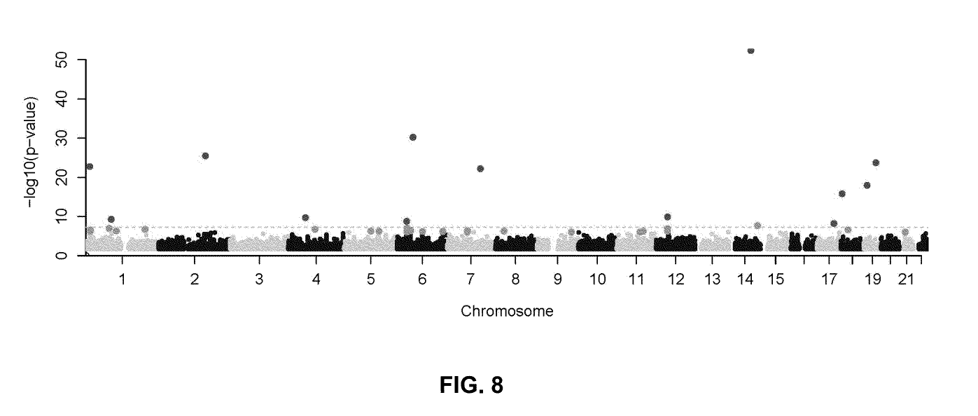

For the zolendronate-specific analysis, a subset of 219 cases, comprising subjects who were treated with zolendronate, was analyzed. The control group comprised 2509 samples. Table 8 shows the SNPs found to be the most strongly associated with zolendronate-induced ONJ. FIG. 8 is a Manhattan plot summarizing the results of the WGA study for zolendronate-specific ONJ cases.

TABLE 8

| | | Position (NCBI | | |
|---|---|---|---|---|
| SNP Name | Chromosome | Build 37) | p-value | Odds Ratio |
| rs10874639 | 1 | 1.03E+08 | 5.05E−07 | 1.711 |
| rs11803759 | 1 | 14665131 | 4.85E−07 | 3.49 |
| rs12037134 | 1 | 2.01E+08 | 2.05E−07 | 1.969 |
| rs12093888 | 1 | 14654587 | 2.97E−07 | 3.586 |
| rs1339362 | 1 | 14634053 | 5.81E−07 | 3.184 |
| rs17346571 | 1 | 12835168 | 1.88E−23 | 5.464 |
| rs3768235 | 1 | 85733374 | 5.44E−10 | 2.899 |
| rs638335 | 1 | 78527876 | 1.16E−07 | 1.768 |
| rs78750384 | 2 | 1.58E+08 | 3.67E−26 | 5.711 |
| rs13111701 | 4 | 90212077 | 2.07E−07 | 3.687 |
| rs2228991 | 4 | 57796900 | 2.03E−10 | 3.851 |
| rs41311333 | 5 | 89914925 | 5.68E−07 | 2.35 |
| rs79272398 | 5 | 1.18E+08 | 5.94E−07 | 3.042 |
| rs112430285 | 6 | 32583880 | 9.76E−07 | 2.18 |
| rs114284967 | 6 | 32595682 | 7.18E−07 | 2.617 |
| rs114457175 | 6 | 32596145 | 8.08E−07 | 2.608 |
| rs114621312 | 6 | 44743924 | 3.55E−07 | 3.586 |
| rs115088603 | 6 | 44734793 | 3.55E−07 | 3.586 |
| rs116369462 | 6 | 31253866 | 1.77E−09 | 2.062 |
| rs117794673 | 6 | 44755958 | 5.18E−07 | 3.513 |
| rs147204463 | 6 | 1.54E+08 | 7.60E−07 | 3.055 |
| rs147773060 | 6 | 32584894 | 9.33E−08 | 1.997 |
| rs150586103 | 6 | 44750126 | 3.55E−07 | 3.586 |
| rs2397118 | 6 | 52701143 | 6.42E−31 | 6.798 |
| rs4367345 | 6 | 85292590 | 6.86E−07 | 3.536 |
| rs4616962 | 6 | 85301754 | 6.86E−07 | 3.536 |
| rs76082037 | 6 | 1.54E+08 | 6.09E−07 | 2.703 |
| rs115636101 | 7 | 67036844 | 4.43E−07 | 3.469 |
| rs116047343 | 7 | 67035722 | 4.43E−07 | 3.469 |
| rs139255097 | 7 | 67063702 | 8.83E−07 | 1.989 |
| rs143435346 | 7 | 67038023 | 8.38E−07 | 3.354 |
| rs147763525 | 7 | 67038980 | 5.11E−07 | 3.439 |
| rs2215137 | 7 | 67051527 | 9.48E−07 | 1.982 |
| rs3757645 | 7 | 1.11E+08 | 6.75E−23 | 6.033 |
| rs56244670 | 7 | 67031366 | 6.01E−07 | 3.41 |
| rs59297873 | 7 | 67028754 | 6.01E−07 | 3.41 |
| rs6460379 | 7 | 67023716 | 6.01E−07 | 3.41 |
| rs6460380 | 7 | 67023888 | 6.01E−07 | 3.41 |
| rs6460381 | 7 | 67023889 | 6.01E−07 | 3.41 |
| rs6954375 | 7 | 67024682 | 6.01E−07 | 3.41 |
| rs73135794 | 7 | 67068034 | 7.63E−07 | 1.997 |
| rs73699812 | 7 | 67015206 | 6.01E−07 | 3.41 |
| rs73699819 | 7 | 67024919 | 6.01E−07 | 3.41 |
| rs73699820 | 7 | 67027257 | 6.01E−07 | 3.41 |
| rs73699821 | 7 | 67033226 | 8.90E−07 | 3.342 |
| rs73699822 | 7 | 67038297 | 6.21E−07 | 3.401 |
| rs73699823 | 7 | 67038355 | 6.21E−07 | 3.401 |
| rs76096884 | 7 | 67022809 | 4.33E−07 | 3.476 |
| rs7786958 | 7 | 67024095 | 6.01E−07 | 3.41 |
| rs78162026 | 7 | 67041089 | 6.26E−07 | 3.4 |
| rs78764728 | 7 | 67027163 | 6.01E−07 | 3.41 |
| rs79740765 | 7 | 67029749 | 6.01E−07 | 3.41 |
| rs80310036 | 7 | 67020447 | 4.33E−07 | 3.476 |
| rs117243673 | 8 | 32717709 | 5.92E−07 | 5.153 |
| rs112945013 | 9 | 1.16E+08 | 9.90E−07 | 1.919 |
| rs10765320 | 11 | 90204966 | 6.56E−07 | 0.02919 |
| rs4529900 | 11 | 79173703 | 8.98E−07 | 1.782 |
| rs112344249 | 12 | 38091874 | 6.13E−07 | 2.737 |
| rs11543410 | 12 | 38219455 | 1.30E−10 | 2.924 |
| rs73097853 | 12 | 38103332 | 1.33E−07 | 2.846 |
| rs11628901 | 14 | 96010424 | 2.28E−08 | 1.95 |
| rs17182244 | 14 | 73726151 | 4.66E−53 | 11.38 |
| rs12602205 | 17 | 56232675 | 6.43E−09 | 2.255 |
| rs1791084 | 18 | 3339405 | 1.73E−16 | 2.871 |
| rs9965541 | 18 | 23436642 | 2.69E−07 | 0.314 |
| rs117389731 | 19 | 40540697 | 1.98E−24 | 12.14 |
| rs2304255 | 19 | 10475649 | 1.19E−18 | 3.301 |
| rs75682881 | 21 | 18775707 | 9.22E−07 | 2.725 |

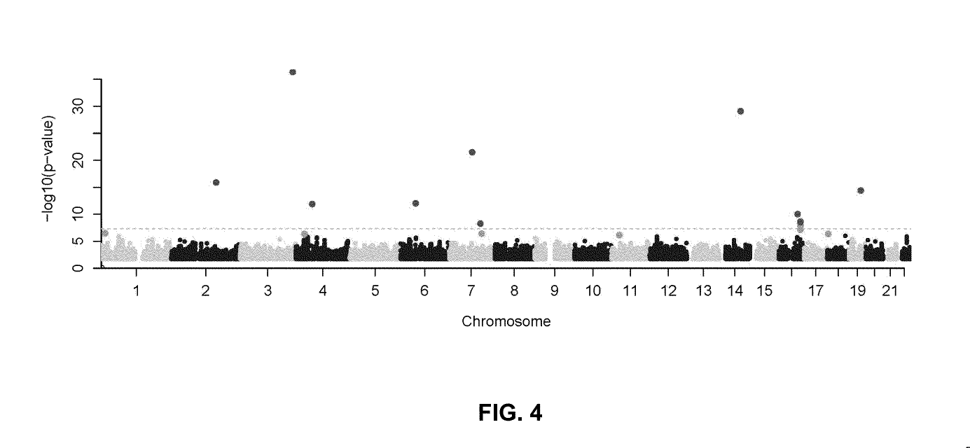

For the alendronate-specific analysis, a subset of 94 cases, comprising subjects who were treated with alendronate, was analyzed. The control group comprised 2509 samples. Table 9 shows the SNP found to be the most strongly associated with alendronate-induced ONJ. FIG. 4 is a Manhattan plot summarizing the results of the WGA study for alendronate-specific ONJ cases.

TABLE 9

| | | Position (NCBI | | |
|---|---|---|---|---|
| SNP Name | Chromosome | Build 37) | p-value | Odds Ratio |
| rs17346571 | 1 | 12835168 | 3.56E−07 | 3.814 |
| rs78750384 | 2 | 157854428 | 1.41E−16 | 6.552 |
| rs6795668 | 3 | 186417883 | 4.71E−37 | 48.39 |
| rs142535692 | 4 | 29946173 | 4.81E−07 | 5.919 |
| rs2228991 | 4 | 57796900 | 1.37E−12 | 6.08 |
| rs2397118 | 6 | 52701143 | 1.02E−12 | 5.202 |
| rs10250411 | 7 | 82348283 | 3.44E−22 | 6.181 |
| rs3757645 | 7 | 111426682 | 5.86E−09 | 4.37 |
| rs78841495 | 7 | 115780254 | 4.31E−07 | 4.981 |
| rs11030099 | 11 | 27677583 | 8.00E−07 | 0.1291 |
| rs17182244 | 14 | 73726151 | 8.72E−30 | 11.8 |
| rs117348856 | 16 | 65966090 | 9.92E−11 | 11.49 |
| rs117798852 | 16 | 75894335 | 2.48E−09 | 7.862 |
| rs139515455 | 16 | 75777752 | 7.94E−08 | 6.687 |
| rs142106800 | 16 | 75969590 | 1.36E−08 | 6.961 |
| rs149490018 | 16 | 75677236 | 3.23E−09 | 7.762 |
| rs1791084 | 18 | 3339405 | 4.62E−07 | 2.45 |
| rs117389731 | 19 | 40540697 | 4.33E−15 | 11.95 |

Dimensions ONJ Cases vs. Controls

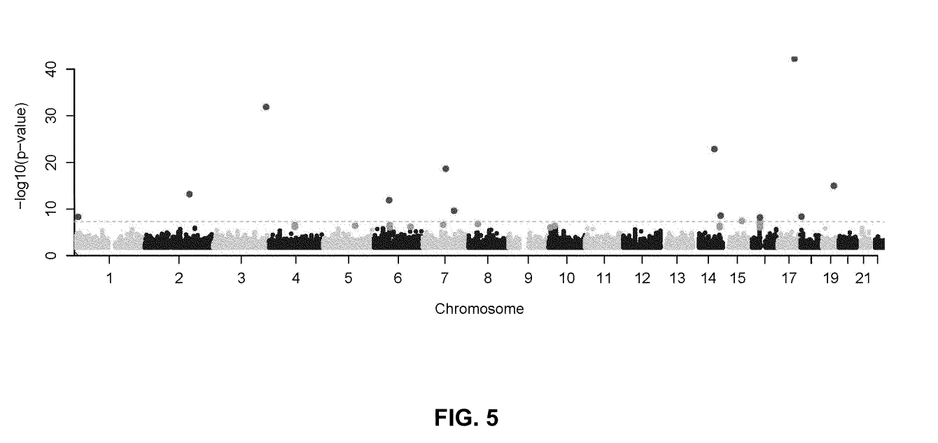

For the Dimension phenotype analysis, a subset of 87 cases, comprising subjects who had a necrosis (ADR) larger than 2.5 cm, was analyzed. The control group comprised 2509 samples. Table 10 shows the SNPs found to be the most strongly associated with Dimensions ONJ. FIG. 5 is a Manhattan plot summarizing the results of the WGA study for Dimensions ONJ cases.

TABLE 10

| | | Position (NCBI | | |
|---|---|---|---|---|
| SNP Name | Chromosome | Build 37) | p-value | Odds Ratio |
| rs17346571 | 1 | 12835168 | 5.29E−09 | 4.499 |
| rs78750384 | 2 | 1.58E+08 | 6.87E−14 | 5.833 |
| rs6795668 | 3 | 1.86E+08 | 1.39E−32 | 39.98 |
| rs13111701 | 4 | 90212077 | 2.79E−07 | 5.277 |
| rs35282873 | 4 | 90191457 | 6.81E−07 | 5.245 |
| rs35855515 | 4 | 90191450 | 5.76E−07 | 5.305 |
| rs1482370 | 5 | 1.13E+08 | 4.11E−07 | 0.3781 |
| rs145566965 | 6 | 1.28E+08 | 7.18E−07 | 9.836 |
| rs16886072 | 6 | 54727815 | 6.21E−07 | 2.827 |
| rs16886073 | 6 | 54729255 | 5.70E−07 | 2.837 |
| rs16886088 | 6 | 54731868 | 5.61E−07 | 2.838 |
| rs16886105 | 6 | 54735344 | 5.61E−07 | 2.838 |
| rs1910352 | 6 | 54736132 | 4.90E−07 | 2.853 |
| rs2397118 | 6 | 52701143 | 1.35E−12 | 5.592 |
| rs56804573 | 6 | 54744397 | 3.16E−07 | 2.981 |
| rs6911699 | 6 | 54739073 | 9.14E−07 | 2.825 |
| rs73437775 | 6 | 54726908 | 5.78E−07 | 2.835 |
| rs73437777 | 6 | 54726979 | 5.81E−07 | 2.835 |
| rs73437783 | 6 | 54730212 | 5.61E−07 | 2.838 |
| rs73437784 | 6 | 54730969 | 5.61E−07 | 2.838 |
| rs73437785 | 6 | 54731279 | 5.61E−07 | 2.838 |
| rs73437796 | 6 | 54741458 | 9.11E−07 | 2.826 |
| rs75768710 | 6 | 54728789 | 5.70E−07 | 2.837 |
| rs7739951 | 6 | 54732039 | 5.61E−07 | 2.838 |
| rs10250411 | 7 | 82348283 | 2.44E−19 | 5.986 |
| rs2353064 | 7 | 72752461 | 2.50E−07 | 0.2628 |
| rs3757645 | 7 | 1.11E+08 | 2.56E−10 | 5.151 |
| rs148942665 | 8 | 36680564 | 1.76E−07 | 7.168 |
| rs11254835 | 10 | 6915748 | 9.41E−07 | 2.723 |
| rs7098605 | 10 | 21635195 | 4.34E−07 | 2.44 |
| rs11628901 | 14 | 96010424 | 2.87E−09 | 2.752 |
| rs116976498 | 14 | 92477244 | 8.85E−07 | 7.442 |
| rs117557425 | 14 | 92444705 | 5.74E−07 | 7.799 |
| rs138588020 | 14 | 92500938 | 4.59E−07 | 7.974 |
| rs139878988 | 14 | 92457977 | 5.68E−07 | 7.805 |
| rs141781242 | 14 | 92456666 | 5.68E−07 | 7.805 |
| rs17182244 | 14 | 73726151 | 1.48E−23 | 9.492 |
| rs186096368 | 14 | 92508211 | 4.57E−07 | 7.977 |
| rs75263479 | 14 | 92446688 | 5.71E−07 | 7.802 |
| rs2572217 | 15 | 66062227 | 3.88E−08 | 2.658 |
| rs3024608 | 16 | 27363686 | 5.05E−08 | 3.812 |
| rs3024612 | 16 | 27364233 | 6.74E−09 | 4.061 |
| rs3024614 | 16 | 27364345 | 7.08E−09 | 4.053 |
| rs3024620 | 16 | 27364918 | 2.41E−07 | 3.627 |
| rs3024629 | 16 | 27366030 | 1.23E−07 | 3.799 |
| rs3024648 | 16 | 27367999 | 9.26E−07 | 3.406 |
| rs61748812 | 17 | 59668517 | 6.11E−43 | 29.77 |
| rs1791084 | 18 | 3339405 | 4.55E−09 | 2.992 |
| rs117389731 | 19 | 40540697 | 1.15E−15 | 13.25 |

Latency ONJ Cases vs. Controls

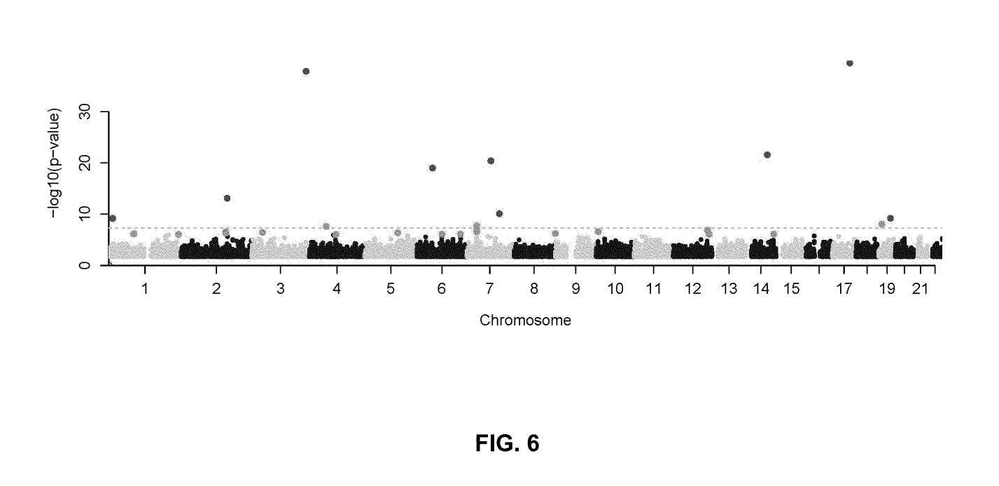

For the Latency phenotype analysis, a subset of 85 cases, comprising subjects who had the who had the ADR onset before 21 months from the starting treatment, was analyzed. The control group comprised 2509 samples. Table 11 shows the SNPs found to be the most strongly associated with Dimensions ONJ. FIG. 6 is a Manhattan plot summarizing the results of the WGA study for Dimensions ONJ cases.

TABLE 11

| | | Position (NCBI | | |
|---|---|---|---|---|
| SNP Name | Chromosome | Build 37) | p-value | Odds Ratio |
| rs17346571 | 1 | 12835168 | 7.19E−10 | 4.884 |
| rs2689167 | 1 | 2.39E+08 | 9.68E−07 | 2.869 |
| rs3768235 | 1 | 85733374 | 6.86E−07 | 3.409 |
| rs142828915 | 2 | 1.53E+08 | 4.95E−07 | 7.857 |
| rs145679681 | 2 | 1.53E+08 | 3.18E−07 | 8.21 |
| rs78750384 | 2 | 1.58E+08 | 8.05E−14 | 5.834 |
| rs80175539 | 2 | 1.53E+08 | 2.96E−07 | 8.274 |
| rs149204177 | 3 | 36671233 | 3.75E−07 | 9.932 |
| rs6795668 | 3 | 1.86E+08 | 1.43E−38 | 53.23 |
| rs2228991 | 4 | 57796900 | 2.63E−08 | 5.022 |
| rs79583356 | 4 | 90899414 | 9.56E−07 | 4.773 |
| rs1482370 | 5 | 1.13E+08 | 4.56E−07 | 0.3734 |
| rs2397118 | 6 | 52701143 | 1.07E−19 | 8.067 |
| rs4367345 | 6 | 85292590 | 8.66E−07 | 4.999 |
| rs4616962 | 6 | 85301754 | 8.66E−07 | 4.999 |
| rs73013023 | 6 | 1.48E+08 | 8.29E−07 | 3.736 |
| rs10215226 | 7 | 33633443 | 1.87E−07 | 4.664 |
| rs10223962 | 7 | 33624926 | 2.04E−07 | 4.639 |
| rs10250411 | 7 | 82348283 | 4.16E−21 | 6.676 |
| rs10257595 | 7 | 33626329 | 1.66E−07 | 4.772 |
| rs10277926 | 7 | 33629413 | 2.15E−07 | 4.623 |
| rs140522128 | 7 | 33615962 | 2.01E−08 | 5.408 |
| rs1971893 | 7 | 33642201 | 1.93E−07 | 4.654 |
| rs3757645 | 7 | 1.11E+08 | 8.38E−11 | 5.449 |
| rs60406451 | 7 | 33615177 | 2.01E−07 | 4.643 |
| rs6966869 | 7 | 33643403 | 1.94E−07 | 4.653 |
| rs73321074 | 7 | 33618844 | 2.50E−07 | 4.58 |
| rs79252669 | 7 | 33628694 | 1.87E−07 | 4.668 |
| rs190581844 | 8 | 1.45E+08 | 6.30E−07 | 7.19 |
| rs11254835 | 10 | 6915748 | 2.79E−07 | 2.784 |
| rs10270 | 12 | 1.23E+08 | 8.92E−07 | 2.183 |
| rs113798063 | 12 | 1.17E+08 | 1.33E−07 | 4.967 |
| rs114351729 | 14 | 95860797 | 8.41E−07 | 4.986 |
| rs17182244 | 14 | 73726151 | 2.86E−22 | 9.156 |
| rs61748812 | 17 | 59668517 | 3.53E−40 | 26.23 |
| rs117389731 | 19 | 40540697 | 6.60E−10 | 8.409 |
| rs2304255 | 19 | 10475649 | 1.01E−08 | 3.12 |

### REFERENCES

Sambrook et al., Molecular Cloning, Cold Spring Harbor Laboratory Press, Cold Spring Harbor, N.Y., 1989.
Innis et al., Proc. Natl. Acad. Sci. USA, 85(24): 9436-9449, 1988.
Guilfoyle et al., Nucleic Acids Research, 25: 1854-1858, 1997.
Walker et al., Proc. Natl. Acad. Sci. USA, 89: 392-396, 1992.
Kwoh et al., Proc. Natl. Acad. Sci. USA, 86: 1173, 1989.
Frohman, PCR Protocols: A Guide to Methods and Applications, Academic Press, N.Y., 1990.
Ohara et al., Proc. Natl. Acad. Sci. USA, 86: 5673-5677, 1989.

1. A method of identifying a subject afflicted with, or at risk of, developing osteonecrosis of the jaw (ONJ), the method comprising:
(a) obtaining a nucleic acid-containing sample from the subject; and
(b) analyzing the sample to detect the presence of at least one genetic marker, wherein the presence of the at least one genetic marker indicates that the subject is afflicted with, or at risk of, developing ONJ.

2. The method of claim 1, wherein the genetic marker is any of alleles, microsatellites, SNPs, or haplotypes.
